(19) United States
(12) Patent Application Publication
(10) Pub. No.: US 2012/0289492 A1
(43) Pub. Date: Nov. 15, 2012

(54) METHODS OF TREATING MITOCHONDRIAL DISORDERS USING METALLOPORPHYRINS

(75) Inventors: Manisha Patel, Englewood, CO (US); Li-Ping Liang, Aurora, CO (US)

(73) Assignee: The Regents of the University of Colorado, a body corporate, Denver, CO (US)

(21) Appl. No.: 13/393,788

(22) PCT Filed: Sep. 2, 2010
(86) PCT No.: PCT/US10/47723

§ 371 Date: Apr. 11, 2012

Related U.S. Application Data
(60) Provisional application No. 61/239,293, filed on Sep. 2, 2009.

(51) Int. Cl. A61K 31/555; A61P 25/08

(52) U.S. Cl. 514/185

(57) ABSTRACT

Methods of treating a mitochondrial disorder including epilepsy, a neurological disorder, an inherited mitochondrial disease or inherited epilepsies, a pediatric epilepsy, an encephalopathy or a pediatric movement disorder are provided, as well as compounds useful in the methods of the invention, such as metalloporphyrin compounds as disclosed herein.

### CROSS-REFERENCES TO RELATED APPLICATIONS

This application claims the benefit of U.S. Provisional Patent Application No. 61/239,293, filed on Sep. 2, 2009, the contents of which are incorporated herein in its entirety for all purposes.

### STATEMENT AS TO RIGHTS TO INVENTIONS MADE UNDER FEDERALLY SPONSORED RESEARCH OR DEVELOPMENT

This invention was made with government support under grant number R21NS053548 awarded by the National Institutes of Health. The Government has certain rights in the invention.

### BACKGROUND OF THE INVENTION

Epilepsies are a group of clinical syndromes that affect more than 50 million people worldwide. Animals are also known to be affected by these syndromes. The incidence of epilepsy is high in children younger than 5 years of age and in individuals older than 65 years. Epileptic seizures are the most common feature observed in children with inherited mitochondrial diseases. Therefore, there is a need for treating epilepsies such as treating epileptic seizures. Provided herein are methods and compositions for meeting these and other needs in the art.

### BRIEF SUMMARY OF THE INVENTION

Provided herein, inter alia, are novel methods of treating a mitochondrial disorder comprising administering to a subject in need thereof a therapeutically effective amount of a metalloporphyrin compound.

In another aspect, provided herein are metalloporphyrin compounds useful for methods of treating a mitochondrial disorder. In some embodiments, the metalloporphyrin compound has the formula:

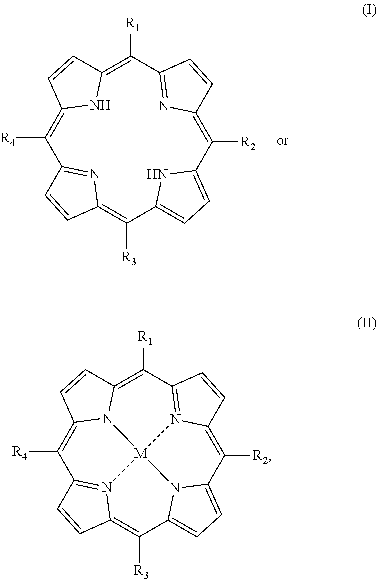

wherein R₁, R₂, R₃, and R₄ are each independently —CF₃, —CO₂R₈, —COR_{8′},

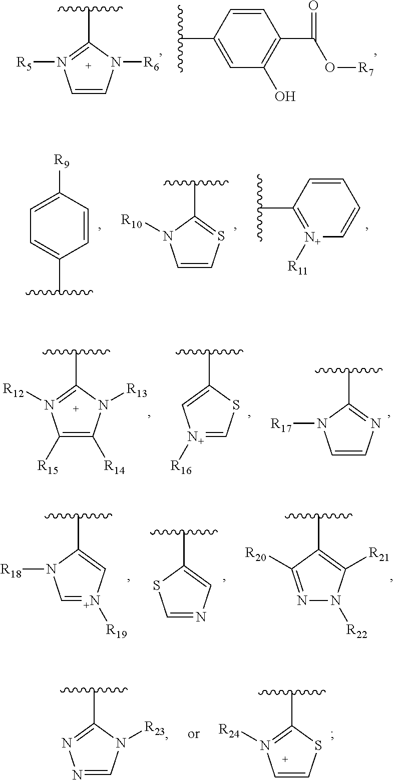

R₅, R₆, R₇, R₈, R_{8′}, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄ are each independently hydrogen, halogen, —CN, —CF₃, —OH, —NH₂, —COOH, —COOR₂₅, —CH₂COOR₂₅, —CH₂COOH, an unsubstituted or substituted alkyl, unsubstituted or substituted heteroalkyl, unsubstituted or substituted cycloalkyl, unsubstituted or substituted heterocycloalkyl, unsubstituted or substituted aryl, or an unsubstituted or substituted heteroaryl; R₂₅ is an unsubstituted alkyl; and M is a metal. In some embodiments, R₂₅ is C₁₋₁₀ alkyl. In some embodiments, R₂₅ is —CH₃ or a C₁₋₅ alkyl. In some embodiments, the metal is manganese, iron, cobalt, copper, nickel, or zinc.

In some embodiments, R₁, R₂, R₃, and R₄ are

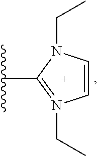

and the metal is manganese. In some embodiments, R₁ and R₃ are —CO₂—CH₃, R₂ and R₄ are —CF₃, and the metal is manganese. In some embodiments, R₁ and R₃ are

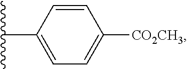

### R₂ and R₄ are

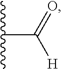

and the metal is manganese.

### BRIEF DESCRIPTION OF THE DRAWINGS

FIG. 1: Exemplary metalloporphyrin compounds and various parameters of some of the compounds.

FIG. 2: An exemplary histogram of the number and duration of seizures in monitored mice.

FIG. 3: Exemplary concentrations of a metalloporphyrin compounds in plasma (3A) or brain (3B) of mice at different time points. 3C represents an exemplary histogram of a metalloporphyrin compound in mouse forebrain fractions.

FIG. 4: Exemplary experiments of effects of a metalloporphyrin compound on the inter-seizure interval (A) or total number of seizures (B).

FIG. 5: Exemplary histograms of levels of various compounds in mice in the presence or absence of a metalloporphyrin compound namely: aconitase (A), ATP (B), 3-nitrotrysosine formation (C), CoASH (D) and Na+—K+ ATPase activity levels (E).

FIG. 6: Exemplary histograms of effects of AEOL11207 on kainate-induced chronic epilepsy development and oxidative stress in rats.

FIG. 7: Survival of Sod2−/− mice treated with AEOL 11207 or vehicle was analyzed by a Kaplan-Meier survival curve. Lifespan from 72 vehicle and 21 AEOL 11207 treated Sod2−/−mice was analyzed. *p<0.01 vehicle vs AEOL11207.

FIG. 8: The duration of averaged spontaneous seizures (A) from vehicle-treated Sod2−/− mice from 16 to 20 days old. Bars represent mean+S.E.M, *p<0.05, **p<0.01 compared to 20 days old, one way ANOVA, n=9-51 per group. Total number (B), frequency (C) and duration (D) of spontaneous seizures observed after the second week of post-natal life with vehicle or AEOL11207-treated Sod2−/− mice. Bars represent mean+S.E.M, *p<0.01 vs. vehicle treatment Sod2 −/− mice; student test, n=23-146 per group.

FIG. 9: Panel 1: Representative H&E and Fluoro-jade B staining images in the parietal cortex of Sod2 −/− mice at 15-16 days old with vehicle or AEOL11207 treatment. H&E staining (A, B, C) and Fluoro jade B staining (D, E, F). Control (A, D), vehicle (B, E) and AEOL11207 (C, F). The insets on the upper right corner of each picture are the enlarged image from the white rectangle. Panel 2: Quantitative analysis of Fluoro jade B fluorescence in the parietal cortex of Sod2 −/− mice at 15-16 days old with vehicle or AEOL11207 treatment. Bars represent mean+S.E.M, *P<0.01 vs wild type with same treatment; #p<0.05 vs. vehicle treatment Sod2 −/− mice; two way ANOVA, n=6 mice per group.

FIG. 10: CoASH (A); CoASSG (B); CoASH/CoASSG ratios (C); aconitase activity (D); 3-nitrotyrosine (E); cysteine and methionine (G) in mitochondrial fractions of Sod2 mutant mice after 15-16 day of birth with vehicle or AEOL11207 treatment. Bars represent mean+S.E.M, *P<0.01 vs wild type with same treatment; #p<0.05 vs. vehicle treatment Sod2 −/− mice; two-way ANOVA, n=6-12 mice per group.

FIG. 11: Panel A: Representative Glutamate transporter GLT1 Western blot images in the hippocampus of Sod mutant mice at 15-16 days old with vehicle or AEOL11207 treatment. Panel B: Glutamate transporter GLT1 protein density was assessed by Western blot analysis in hippocampi of in the hippocampus of Sod2 −/− mice at 15-16 days old with vehicle or AEOL11207 treatment. Each value was normalized with β-actin. Data were expressed as a percent control using vehicle treatment Sod2+/+ as controls (100%). Bars represent mean +S.E.M, *P<0.01 vs wild type with same treatment; #p<0.05 vs. vehicle treatment Sod2 −/− mice; two way ANOVA, n=5 mice per group.

FIG. 12: ATP production (A) and Na⁺, K⁺ATPase activity(B) in forebrain of Sod2 mutant mice after 15-16 day of birth with vehicle or AEOL11207 treatment. Bars represent mean+S.E.M, *P<0.01 vs wild type with same treatment; #p<0.05 vs. vehicle treatment Sod2 −/− mice; two-way ANOVA, n=6-12 mice per group.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″′)═NR″″, —NR—C(NR′R″)═NR″″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R″′, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —S—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (0), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, -NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “ size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “ lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “effective amount” or “therapeutically effective amount” refers to the amount of an active agent sufficient to induce a desired biological result. That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

A “subject,” “individual,” or “patient,” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vitro or cultured in vitro are also encompassed. In some embodiments, the subject or patient is a child. In some embodiments, the subject or patient is a young child. In some embodiments, the subject or patient is an infant.

As defined herein, the term “child” or “children” as used herein means persons over the age of 3 years and prior to adolescence. As used herein, the term “young child” or “young children” means persons from the age of more than 12 months up to the age of three years. As used herein, the term “infant” means a person not more than 12 months of age.

### II. Methods

Provided herein are methods and compositions for treating a mitochondrial disorder in a subject. The method includes administering to a subject in need thereof a therapeutically effective amount of a metalloporphyrin compound. As used herein, the term mitochondrial disorder and mitochondrial dysfunction can be used interchangeably. Compositions contemplated herein include, but are not limited to, metalloporphyrin compounds or metalloporphyrin catalytic antioxidant compositions as set forth in Section II below. In some embodiments, the mitochondrial disorder is epilepsy. The subject may have temporal lobe epilepsy or other acquired epilepsies comprising acute or chronic epilepsies arising from pathological insult. In some embodiments, the epilepsy is an acute or chronic epilepsy. The acute or chronic epilepsies may arise from hypoxia, trauma, viral infections, fever, alcohol withdrawal or aging which increase oxidative stress and mitochondrial disorder. In some embodiments, the method reduces the frequency or severity of epileptic seizures of said subject. In some embodiments, the mitochondrial disorder is an acute or chronic neurological disorder. In some embodiments, the subject has an inherited mitochondrial disease or inherited epilepsies.

In some embodiments, the subject is a child or a young child. The subject may have a pediatric epilepsy, encephalopathy or pediatric movement disorder. In some embodiments, the pediatric movement disorder is derived from fever, trauma, metabolic deficiencies, genetic abnormalities, chromosomal abnormalities, hypoxic/ischemic episodes or a combination thereof.

In some embodiment, the mitochondrial disorder is selected from the group consisting of a mitochondrial disease; Myoclonic Epilepsy with Ragged Red Fibers (MERRF); Mitochondrial Myopathy, Encephalopathy, Lactacidosis, and Stroke (MELAS); Maternally Inherited Diabetes and Deafness (MIDD), Leber's Hereditary Optic Neuropathy (LHON); chronic progressive external ophthalmoplegia (CPEO); Leigh Disease; Kearns-Sayre Syndrome (KSS); Friedreich's Ataxia (FRDA); Co-Enzyme Q1O (CoQ1O) Deficiency; Complex I Deficiency; Complex II Deficiency; Complex III Deficiency; Complex IV Deficiency; Complex V Deficiency; other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's Disease; mood disorders; schizophrenia; bipolar disorder; age-associated diseases; cerebral vascular diseases; macular degeneration; diabetes; cancer. In some embodiment, the mitochondrial disorder is a mitochondrial respiratory chain disorder, e.g., a respiratory protein chain disorder. In some embodiment, the disorder is CoQ1O deficiency.

In many cases, a mitochondrial disorder is passed genetically from parent to child (inheritance). In some embodiment, the mitochondrial disorder is selected from the group consisting of inherited mitochondrial diseases or inherited epilepsies.

In some embodiments, the compounds described herein are administered to subjects affected with a pervasive development disorder such as Autistic Disorder, Asperger's Disorder, Childhood Disintegrative Disorder (CDD), Rett's Disorder, and PDD-Not Otherwise Specified (PDD-NOS).

In some embodiments, the mitochondrial disorder is an acute or chronic neurological disorder. Provided herein are methods of treating such acute or chronic neurological disorders by administering to a subject a therapeutically effective amount of a metalloporphyrin composition, e.g., metalloporphyrin catalytic antioxidants.

In some embodiments, the mitochondrial disorder is an acute or chronic epilepsy, e.g., an acute or chronic epilepsy arising from pathological insult. Some examples of this form of epilepsy include, but are not limited to temporal lobe epilepsy and posttraumatic epilepsy. In certain embodiments, acute or chronic epilepsy may arise from hypoxia, trauma, viral infections, agents used for chemical warfare, fever, alcohol withdrawal, aging or combination thereof which increase oxidative stress and mitochondrial disorder.

Mitochondrial disorder is an important therapeutic target for both inherited and acquired epilepsies. Epileptic seizures are the most common feature observed in children with inherited mitochondrial diseases. Mitochondrial oxidative stress have been observed to have a role in resultant dysfunction in seizure-induced brain injury. Acquired epilepsies account for ˜60% and genetic epilepsies account for ˜40% of all epilepsies. Temporal lobe epilepsy is the most common form of acquired epilepsy and often medically intractable. Epileptic seizures are the most common feature observed in children with inherited mitochondrial diseases.

Some embodiments herein concern candidate metalloporphyrins for treating childhood and adult epilepsies. In other embodiments, candidate metalloporphyrins may be used to treat childhood epilepsies including, but not limited to, epilepsies attributed to childhood mitochondrial disease. In certain embodiments, one or more oral doses of compositions contemplated herein may be administered to a child in need of such a treatment.

In some embodiments, a child is treated for epileptic seizures by administering to the child a therapeutically effective amount of a metalloporphyrin composition. In accordance with these embodiments, a child may have an inherited mitochondrial disease.

Certain childhood disorders contemplated herein include, but are not limited to pediatric epilepsies, encephalopathies or pediatric movement disorders. Pediatric movement disorders include, but are not limited to, those derived from fever, trauma, metabolic deficiencies, genetic or chromosomal abnormalities, hypoxic/ischemic episodes or combination thereof.

Some embodiments include treating neuronal disorders in animals with a pharmaceutically acceptable composition disclosed herein, for example, a household pet may be treated.

In some embodiments, the methods provided herein are effective in treating an epileptic seizure. Epileptic seizures are a common phenotype of inherited mitochondrial diseases arising from mitochondrial DNA (mtDNA) mutation/deletion. The best characterized of these diseases is myoclonic epilepsy with ragged red fibers (MERRF), the first epilepsy in which a molecular defect was identified and linked with the epilepsy syndrome. The molecular defect in MERRF arises from a single mutation of the tRNAlys resulting in a disorder consisting of myoclonic epilepsy and a characteristic myopathy with ragged red fibers. Several mitochondrial disorders have been linked to mutations in mitochondrial genes encoded by either the nuclear or mitochondrial genome. The high prevalence of epilepsy among mitochondrial diseases underscores the importance of developing therapies for these disorders. Mitochondrial disorder can be a consequence of acute and chronic seizures. One important by-product of mitochondrial metabolism is the production of reactive oxygen species (ROS). While abundant and overlapping endogenous antioxidants exist to overcome normal cellular ROS production, excessive production of ROS can overwhelm antioxidant defenses resulting in oxidation of vulnerable cellular targets. Using surrogate markers of target oxidation in the kainic acid model, prolonged seizures have been identified that can oxidatively damage mitochondrial DNA, susceptible mitochondrial proteins and cellular lipids. In addition to being an acute consequence of status epilepticus (SE), mitochondrial ROS production re-emerges immediately prior to development of chronic epilepsy assessed by behavioral analysis, suggesting that ROS formation could contribute to epileptogenesis.

Several normal functions of mitochondria, ranging from bioenergetics to metabolic functions, can impact neuronal excitability. These include, but are not limited to, cellular ATP production, ROS formation, synthesis and metabolism of neurotransmitters, fatty acid oxidation, calcium homeostasis and control of apoptotic/necrotic cell death. Vital functions that contribute to the seizures associated with mitochondrial disorder remains unclear. Although mitochondrial encephalopathies due to genetic causes are rare, they may provide important lessons regarding the mechanisms underlying acquired epilepsy such as temporal lobe epilepsy.

Mitochondrial oxidative stress and disorder have been demonstrated as a risk factor for age-related seizures in Sod2−/+ mice. Previous experiments provide experimental evidence linking mitochondrial oxidative stress with increased seizure susceptibility induced by aging, environmental stimulation, or kainate administration. Heterozygous Sod2−/+ mice (B6 background), unlike the homozygous knockouts (Sod2−/−) appear both biochemically and phenotypically normal at birth but develop age-related deficits consistent with chronic mitochondrial oxidative stress. A subset of Sod2−/+ mice developed spontaneous and handling-induced seizures as a function of advancing age. Age-related onset of seizures in Sod2−/+ mice correlated with increased mitochondrial oxidative stress (mitochondrial aconitase inactivation and mitochondrial, but not nuclear DNA oxidation) and mitochondrial disorder as measured by oxygen utilization. Prior to the age at which spontaneous and handling-induced seizures occurred, Sod2−/+ mice showed increased susceptibility to kainate-induced seizures and hippocampal cell loss. This suggests that mitochondrial oxidative stress and resultant dysfunction may be an important mechanism underlying the increased seizure susceptibility in Sod2−/+ mice. Whereas the Sod2−/+ mice are a model of age-related chronic oxidative stress and mitochondrial dysfunction, Sod2−/− mice, disclosed herein, provide a model of acute oxidative stress and mitochondrial dysfunction occurring in early life.

Development of animal models in which epilepsy arises due to mitochondrial dysfunction is a useful tool in understanding the mechanisms of epileptogenesis associated with mitochondrial diseases and oxidative stress and in identifying metalloporphyrins effective in treating epilepsy. Genetically modified mice lacking SOD have provided strong evidence in support of the role of mitochondrial dysfunction and oxidative stress in epilepsy. SOD2 deficient mice demonstrate extensive mitochondrial dysfunction correlating with increased incidence of spontaneous and evoked seizures, described previously. Mitochondrial disease has been characterized in SOD2 deficient mice generated in several background strains. Whereas Sod2−/− (B6 Sod2−/−) mice are embryonic lethal, CD-1 Sod2−/− mice develop and live approximately 8-10 days exhibiting frequent seizures. More recently, Sod2−/− mice bred of a mixed background (DBA/2J X B6D2 or B6D2Sod2−/−) have been generated, which live approximately 3 weeks without pharmacological intervention. In the second week of postnatal life these mice exhibit frequent spontaneous motor seizures. SOD2 deficient mice have been shown to be a powerful tool for demonstrating the efficacy of antioxidants in treating mitochondrial dysfunction and oxidative stress. Therefore, the longer-lived Sod2−/− mice provide a model of epilepsy associated with mitochondrial disease in which therapeutic interventions can be tested.

Compositions and methods herein concern treatments for, including, but not limited to, the following epilepsy disorders: 1) inherited mitochondrial diseases arising from mitochondrial DNA mutation/deletion due to the high prevalence of epilepsy among mitochondrial diseases; 2) pediatric epilepsies, encephalopathies and pediatric movement disorders that arise due to metabolic factors for example, fever, trauma, metabolic deficiencies, genetic or chromosomal abnormalities, hypoxic/ischemic episodes or combination thereof; and 3) temporal lobe epilepsy as well as other acquired acute and chronic epilepsies: arising from pathological insult, e.g., hypoxia, trauma, viral infections, chemicals used for warfare, fever, alcohol withdrawal or aging per se which increase oxidative stress and mitochondrial dysfunction. In certain embodiments, compositions and methods herein can include metalloporphyrin agents or derivatives thereof, alone or in combination with other agents for treating a subject in need of such a treatment.

In certain embodiments, compositions and methods herein may include AEOL11207, alone or in combination with other agents. In other embodiments, AEOL11207, a potent lipophilic catalytic antioxidant or other compositions disclosed herein, can be administered by any mode to a subject in need of such a treatment (e.g. for epileptic seizures). Some embodiments herein contemplate that AEOL11207 may be administered alone or in combination with other agents administered via a s.c. route. In certain embodiments, it is contemplated that administration of AEOL11207 to a subject can decrease oxidative damage and/or attenuate epileptic seizures in a subject. In other embodiments, it is contemplated that administration of metalloporphyrin agents or derivatives thereof to a subject can decrease or prevent epileptic seizure occurrence and/or decrease or prevent epileptic seizure side effects.

In certain embodiments, compositions and methods herein may include AEOL11209, alone or in combination with other agents. In other embodiments, AEOL11209, a potent lipophilic catalytic antioxidant or other compositions disclosed herein, can be administered by any mode to a subject in need of such a treatment (e.g. for epileptic seizures). Some embodiments herein contemplate that AEOL11209 may be administered alone or in combination with other agents administered via a s.c. route. In certain embodiments, it is contemplated that administration of AEOL11209 to a subject can decrease oxidative damage and/or attenuate epileptic seizures in a subject. In other embodiments, it is contemplated that administration of metalloporphyrin agents or derivatives thereof to a subject can decrease or prevent epileptic seizure occurrence and/or decrease or prevent epileptic seizure side effects.

In certain embodiments, compositions and methods herein may include AEOL10150, alone or in combination with other agents. In other embodiments, AEOL 10150, a potent lipophilic catalytic antioxidant or other compositions disclosed herein, can be administered by any mode to a subject in need of such a treatment (e.g. for epileptic seizures). Some embodiments herein contemplate that AEOL10150 may be administered alone or in combination with other agents administered via a s.c. route. In certain embodiments, it is contemplated that administration of AEOL10150 to a subject can decrease oxidative damage and/or attenuate epileptic seizures in a subject. In other embodiments, it is contemplated that administration of metalloporphyrin agents or derivatives thereof to a subject can decrease or prevent epileptic seizure occurrence and/or decrease or prevent epileptic seizure side effects.

### III. Metalloporphyrins

In some embodiments, the metalloporphyrin compound useful in the methods provided herein have the formula:

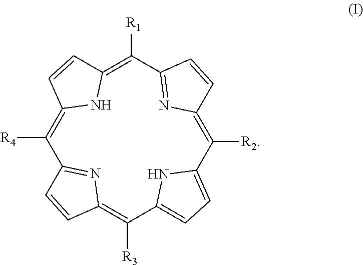

In Formula I, the substituted porphyrin may be bound to a metal. The metal may be manganese, iron, cobalt, copper, nickel, or zinc, including ions thereof. For example, in Formula II, below, M is manganese, iron, cobalt, copper, nickel, or zinc, including ions thereof:

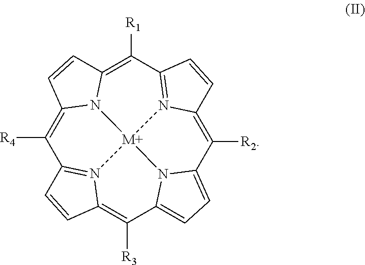

In a specific embodiment, the metal is manganese and has the formula:

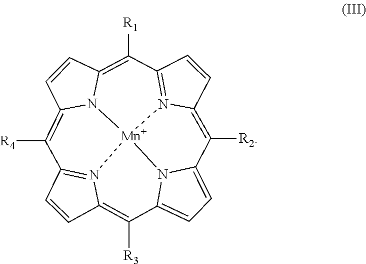

R₁, R₂, R₃, and R₄ may each independently be —CF₃, —CO₂R₈, —COR₈′,

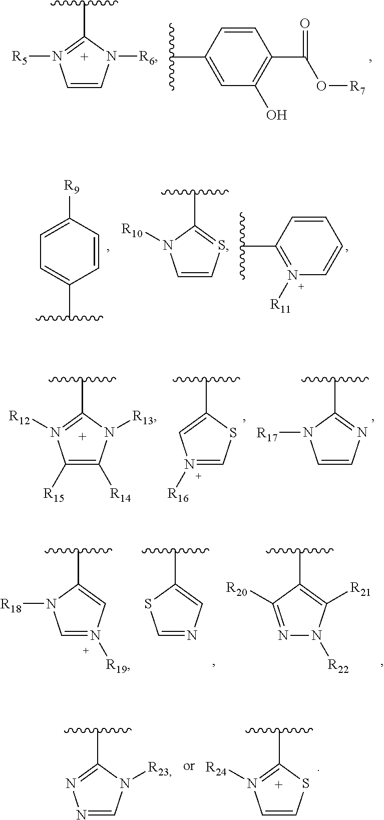

R₁, R₂, R₃, and R₄ may also be

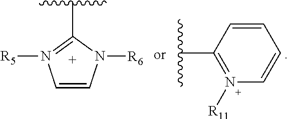

In some embodiments, R₁ and R₃ are independently —CO₂R₈ or —COR_{S}′. R₂ and R₄ may independently be —CF₃ or

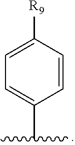

In some related embodiments, R₁ and R₃ are independently —CO₂R₈, and R₂ and R₄ are —CF₃. In other related embodiments, R₁ and R₃ are

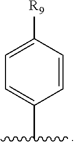

independently —CO₂R₈ and R₂ and R₄ are independently

Where R₁, R₂, R₃, and R₄ contain a positive charge, one of skill will immediately recognize that an anionic compound or molecule will be present where the compound is in solution. Any applicable anionic compound are molecule may be used as a counterion to the positively charges substituents, including for example chloride, fluoride, sulfide, a sulfate, a carbonate, or a phosphate.

Each R₅, R₆, R₇, R₈, R_{8′}, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄ may be the same or different and may each independently be hydrogen, halogen, —CN, —CF₃, —OH, —NH₂, —COOH, —COOR₂₅, —CH₂COOR₂₅, —CH₂COOH, an unsubstituted or substituted alkyl, unsubstituted or substituted heteroalkyl, unsubstituted or substituted cycloalkyl, unsubstituted or substituted heterocycloalkyl, unsubstituted or substituted aryl, or an unsubstituted or substituted heteroaryl. R₂₅ is an unsubstituted alkyl such as C₁₋₁₀ alkyl (e.g., —CH₃ or a C₁₋₅ alkyl). In some embodiments, R₅, R₆, R₇, R₈, R_{8′}, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄ may each independently be hydrogen, halogen, —CN, —CF₃, —OH, —NH₂, —COOH, —COOR₂₅, —CH₂COOR₂₅, —CH₂COOH, substituted or unsubstituted C₁-C₁₀ (e.g., C₁-C₆) alkyl, substituted or unsubstituted 2 to 10 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₈ (e.g., C₅-C₆) aryl, or substituted or unsubstituted 5 to 8 membered (e.g., 5 to 6 membered) heteroaryl. In some embodiments, one or more of R₅, R₆, R₇, R₈, R_{8′}, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄ is unsubstituted. In one embodiment, R₅, R₆, R₇, R₈, R_{8′}, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄ are independently hydrogen or a substituted or unsubstituted C₁-C₁₀ (e.g., C₁-C₆ or C₁-C₃) alkyl.

In one embodiment, R₅, R₆, R₇, R₈, R_{8′}, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄, may independently be hydrogen, halogen, —CN, —CF₃, —OH, —NH₂, —COOH, —COOR₂₅, —CH₂COOR₂₅, —CH₂COOH, R₂₆-substituted or unsubstituted alkyl, R₂₆-substituted or unsubstituted heteroalkyl, R₂₆-substituted or unsubstituted cycloalkyl, R₂₆-substituted or unsubstituted heterocycloalkyl, R₂₆-substituted or unsubstituted aryl, or R₂₆-substituted or unsubstituted heteroaryl. R₂₆ is halogen, —CN, —CF₃, —OH, —NH₂, —COOH, —COOR₂₅, —CH₂COOR₂₅, —CH₂COOH, R₂₇-substituted or unsubstituted alkyl, R₂₇-substituted or unsubstituted heteroalkyl, R₂₇-substituted or unsubstituted cycloalkyl, R₂₇-substituted or unsubstituted heterocycloalkyl, R₂₇-substituted or unsubstituted aryl, or R₂₇-substituted or unsubstituted heteroaryl. In one embodiment, R₂₆ is halogen, —CN, —CF₃, —OH, —NH₂, —COOH, R₂₇-substituted or unsubstituted C₁-C₁₀ (e.g., C₁-C₆) alkyl, R₂₇-substituted or unsubstituted 2 to 10 membered (e.g., 2 to 6 membered) heteroalkyl, R₂₇-substituted or unsubstituted C₃-C₈ (e.g., C₅-C₇) cycloalkyl, R₂₇-substituted or unsubstituted 3 to 8 membered (e.g., 3 to 6 membered) heterocycloalkyl, R₂₇-substituted or unsubstituted C₅-C₈ (e.g., C₅-C₆) aryl, or R₂₇-substituted or unsubstituted 5 to 8 membered (e.g., 5 to 6 membered) heteroaryl.

R₂₇ is halogen, —CN, —CF₃, —OH, —NH₂, —COOH, —COOR₂₅, —CH₂COOR₂₅, —CH₂COOH, R₂₈-substituted or unsubstituted alkyl, R₂₈-substituted or unsubstituted heteroalkyl, R₂₈-substituted or unsubstituted cycloalkyl, R₂₈-substituted or unsubstituted heterocycloalkyl, R₂₈-substituted or unsubstituted aryl, or R₂₈-substituted or unsubstituted heteroaryl. In one embodiment, R₂₇ is halogen, —CN, —CF₃, —OH, —NH₂, —COOH, R₂₈-substituted or unsubstituted C₁-C₁₀ (e.g., C₁-C₆) alkyl, R₂₈-substituted or unsubstituted 2 to 10 membered (e.g., 2 to 6 membered) heteroalkyl, R₂₈-substituted or unsubstituted C₃-C₈ (e.g., C₅-C₇) cycloalkyl, R₂₈-substituted or unsubstituted 3 to 8 membered (e.g., 3 to 6 membered) heterocycloalkyl, R₂₈-substituted or unsubstituted C₅-C₈ (e.g., C₅-C₆) aryl, or R₂₈-substituted or unsubstituted 5 to 8 membered (e.g., 5 to 6 membered) heteroaryl. R₂₈ is halogen, —CN, —CF₃, —OH, —NH₂, —COOH, —COOR₂₅, —CH₂COOR₂₅, —CH₂COOH, unsubstituted alkyl, unsubstituted heteroalkyl, unsubstituted cycloalkyl, unsubstituted heterocycloalkyl, unsubstituted aryl, or unsubstituted heteroaryl.

In one embodiment, R₂₆ and/or R₂₇ are substituted with a substituent group, a size-limited substituent group or a lower substituent group. In another embodiment, R₂₇ and R₂₈ are independently halogen, —CN, —CF₃, —OH, —NH₂, —COON, —COOR₂₅, —CH₂COOR₂₅, —CH₂COOH, unsubstituted C₁-C₁₀ (e.g., C₁-C₆) alkyl, unsubstituted 2 to 10 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₈ (e.g., C₅-C₆) aryl, or unsubstituted 5 to 8 membered (e.g., 5 to 6 membered) heteroaryl.

In a particular embodiment, each R₅, R₆, R₇, R₈, R_{8′}, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, and R₂₅ may be the same or different and may each independently be an alkyl, and particularly a C₁₋₂₀ alkyl, more particularly a C₁₋₁₀ alkyl, and even more particularly a C₁₋₄ alkyl, and even more particularly, a methyl, an ethyl, or a propyl.

In some embodiments, R₈ and R_{8′} are independently hydrogen or an unsubstituted alkyl (e.g. an unsubstituted C₁₋₁₀ alkyl). R_{8′} may also be hydrogen. R₈ may be methyl.

In some embodiments, R₉ is —COOH, —COOR₂₅, —CH₂COOR₂₅, or —CH₂COOH. R₉ may also be —COOR₂₅ or —CH₂COOR₂₅. In certain embodiments, R₉ is —COOR₂₅. In some related embodiments, R₂₅ is an unsubstituted C₁-C₁₀ alkyl, such as methyl.

In a more specific embodiment, R₁ and R₃ may each independently be —CO₂—CH₃, or

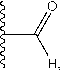

R₂ and R₄ may each independently be —CF₃,

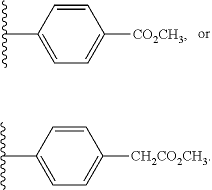

In a specific embodiment, the metalloporphyrin compound of the invention may have the formula:

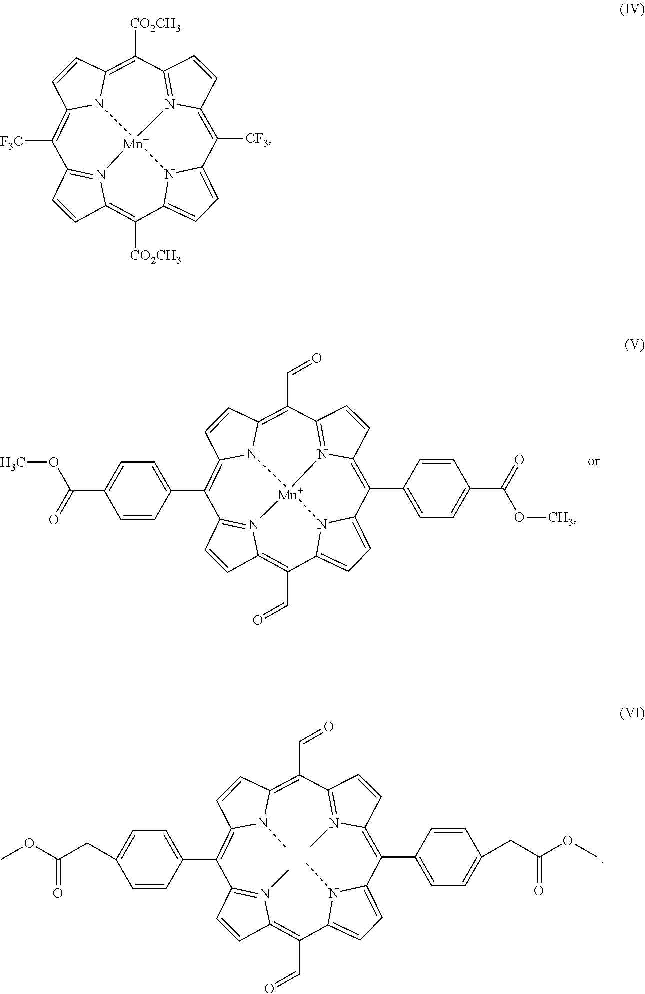

In a another specific embodiment, R₁, R₂, R₃, and R₄ may each independently be

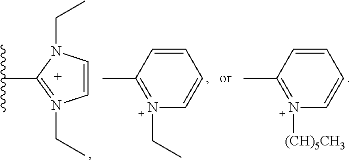

In a specific embodiment, the metalloporphyrin compound of the invention may have the formula:

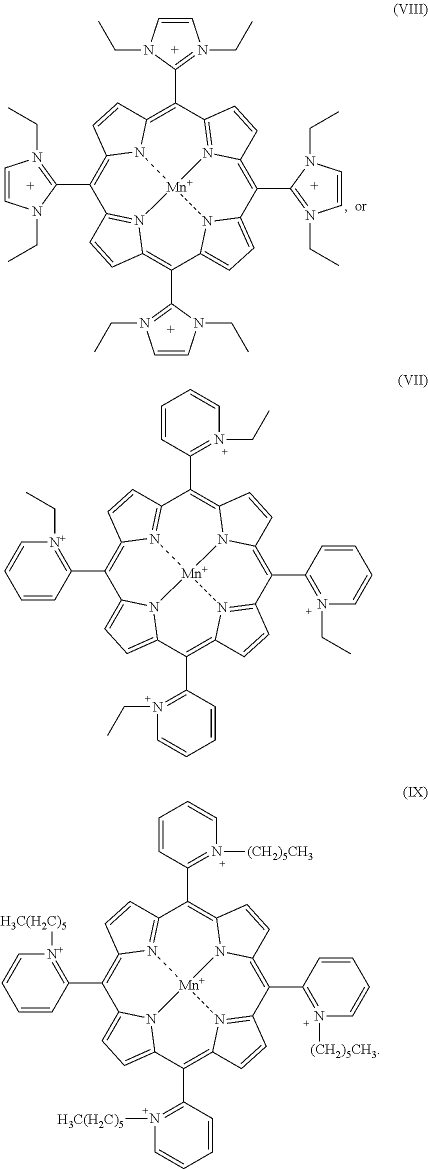

In some embodiments, each substituted group described in the compounds above (e.g., Formulae (I)-(IX I)) is substituted with at least one substituent group. More specifically, in some embodiments, each substituted alkyl, substituted heteroalkyl, substituted cycloalkyl, substituted heterocycloalkyl, substituted aryl, substituted heteroaryl, described in the compounds above (e.g., Formulae (I)-(IX)) are substituted with at least one substituent group. In other embodiments, at least one or all of these groups are substituted with at least one size-limited substituent group. Alternatively, at least one or all of these groups are substituted with at least one lower substituent group.

In other embodiments of the compounds described above (e.g., Formulae (I)-(IX)) each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The compounds described above, metal bound and metal free forms, can be formulated into pharmaceutical compositions suitable for use in the present methods. Such compositions include the active agent (metalloporphyrin compounds) together with a pharmaceutically acceptable carrier, excipient or diluent. The composition can be present in dosage unit form for example, tablets, capsules or suppositories. The composition can also be in the form of a sterile solution, e.g., a solution suitable for injection (e.g., subcutaneous, i.p. or i.v.) or nebulization. Compositions can also be in a form suitable for opthalmic use. The invention also includes compositions formulated for topical administration, such compositions taking the form, for example, of a lotion, cream, gel or ointment. The concentration of active agent to be included in the composition can be selected based on the nature of the agent, the dosage regimen and the result sought. The compounds can also be encapsulated in lysosomes and thereby targeted to enhance delivery.

### IV. Pharmaceutical Compositions

In some embodiments, the metalloporphyrin compounds may from part of a pharmaceutical composition. The pharmaceutical composition may include a metallophorphyrin compound, as disclosed herein, and a pharmaceutically acceptable excipient. A “pharmaceutically acceptable excipient” includes pharmaceutically and physiologically acceptable, organic or inorganic carrier substances suitable for enteral or parenteral administration that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethylcellulose, and polyvinyl pyrrolidone. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the active agent.

In one embodiment, the treatment compound (e.g., metalloporphyrin compounds or metalloporphyrin catalytic antioxidant compositions as set forth in Section II) forms part of a pharmaceutical composition, wherein said pharmaceutical composition comprises said treatment compound and a pharmaceutical acceptable excipient. In one embodiment, the pharmaceutical composition includes a permeabilizer (e.g., a salicylate, a fatty acid, or a metal chelator).

The pharmaceutical composition can be formulated for any route of administration, including enteral, oral, sublingual, buccal, parenteral, ocular, intranasal, pulmonary, rectal, intravaginal, transdermal, and topical routes. Parenteral administration includes, but is not limited to, intravenous, intramuscular, subcutaneous, intradermal, intraperitoneal, intrastemal, intraarterial injection and infusion.

The pharmaceutical composition can be formulated for immediate release or modified release, e.g., modified, sustained, extended, delayed, or pulsatile release, using known methods and excipients.

In one embodiment, the pharmaceutical composition is formulated as a topical composition, an injectable composition, an inhalant, a sustained release composition, or an oral composition. The treatment compound is preferably formulated for parenteral administration, e.g., by subcutaneous injection. If subcutaneous or an alternative type of administration is used, the compounds may be derivatized or formulated such that they have a protracted profile of action.

In another embodiment, the pharmaceutical composition is formulated as a peptide micelle, a targeted micelle, a degradable polymeric dosage form, a porous microsphere, a polymer scaffold, a liposome, or a hydrogel.

The treatment compound may be formulated according to known methods to prepare pharmaceutically useful compositions. An exemplary formulation would be one that is a stable lyophilized product that is reconstituted with an appropriate diluent or an aqueous solution of high purity with optional pharmaceutically acceptable carriers, preservatives, excipients or stabilizers (Remington's Pharmaceutical Sciences 16th edition (1980)). The pharmaceutical composition may include a pharmaceutically acceptable buffer to achieve a suitable pH for stability and for administration.

For parenteral administration, the treatment compound is formulated in a unit dosage injectable form (solution, suspension, or emulsion) with a pharmaceutically acceptable carrier. Preferably, one or more pharmaceutically acceptable anti-microbial agents may be added, such as phenol, m-cresol, and benzyl alcohol.

In one embodiment, one or more pharmaceutically acceptable salts (e.g., sodium chloride), sugars (e.g., mannitol), or other excipients (e.g., glycerin) may be added to adjust the ionic strength or tonicity.

The dosage of the composition of the invention to be administered can be determined without undue experimentation and will be dependent upon various factors including the nature of the active agent (including whether metal bound or metal free), the route of administration, the patient, and the result sought to be achieved. A suitable dosage of mimetic to be administered IV or topically can be expected to be in the range of about 0.01 to 50 mg/kg/day, preferably, 0.1 to 10 mg/kg/day, more preferably 0.1 to 6 mg/kg/day. For aerosol administration, it is expected that doses will be in the range of 0.001 to 5.0 mg/kg/day, preferably, 0.01 to 1 mg/kg/day. Suitable doses will vary, for example, with the compound and with the result sought.

The concentration of mimetic presentation in a solution used to treat cells/tissues/organs in accordance with the methods of the invention can also be readily determined and will vary with the active agent, the cell/tissue/organ and the effect sought.

Certain aspects of the invention can be described in greater detail in the non-limiting Example that follows.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

### Metalloporphyrin Compounds and Metalloporphyrin Catalytic Antioxidant Compositions

In one exemplary method, an animal model was used that exhibits seizures and mitochondrial dysfunction. This model was used to assess lipid soluble metalloporphyrins as potential therapies for catastrophic epilepsies associated with mitochondrial diseases. Mutant cross-bred C57BL6XDBA2F2 (B6D2) mice lacking manganese superoxide dismutase (MnSOD or Sod2), a critical mitochondrial antioxidant, provide such a model. Recently, it was demonstrated that there are unique in vitro biochemical properties and in vivo neuroprotective effects of a novel metalloporphyrin catalytic antioxidant, AEOL11207 (Aeolus Pharmaceuticals Inc., Laguna Niguel, Calif.). It was demonstrated that AEOL11207 attenuates behavioral seizure characteristics of Sod2−/− mice following daily subcutaneous (s.c.) injections beginning postnatal day (PND) 5. This method of delivery is proposed merely to test efficacy of the drug due to limitation of the model and age of animals. It is contemplated herein that certain embodiments include compositions administered orally to a subject.

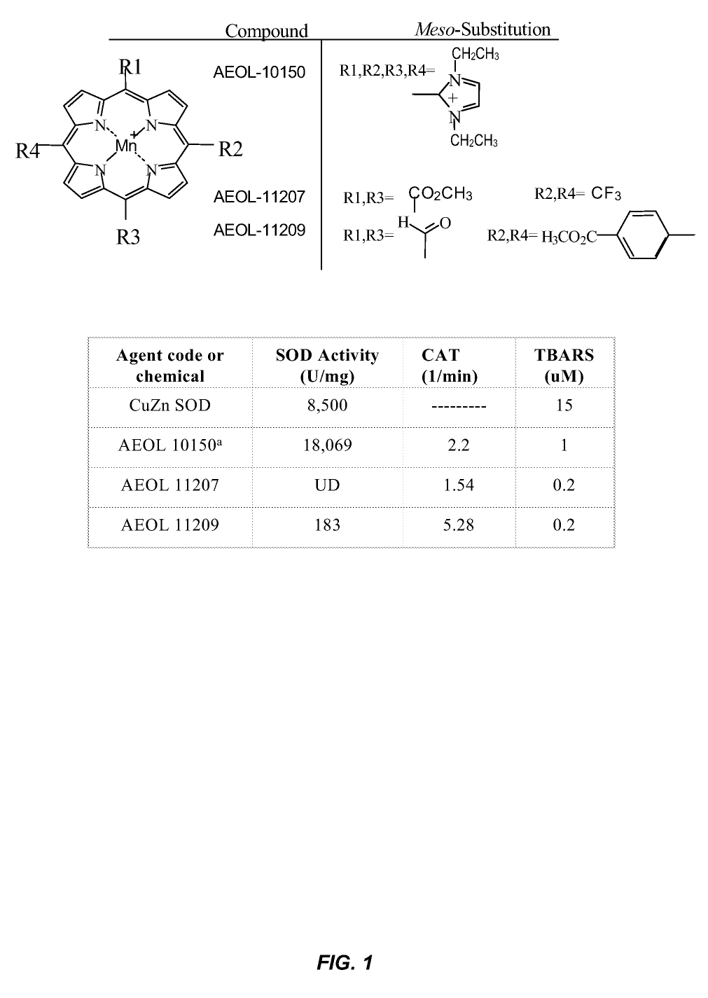

Metalloporphyrin Catalytic Antioxidants: A Unique Class of Molecules for the Potential Treatment of Epilepsies. Catalytic antioxidants are small, molecular mimics of superoxide dismutase and/or catalase, and are also potent detoxifiers of lipid peroxides and peroxynitrite. Because they are catalytic, and not merely free radical scavengers, these compounds are much more potent antioxidants than dietary additives such as vitamin E that act stoichiometrically. FIG. 1 illustrates exemplary structures of representative metalloporphyrin catalytic antioxidants (left). Table (inset) illustrates an exemplary test tube SOD assay: Unit of SOD activity is defined as the amount of compound that inhibits one-half the reduction of cytochrome c by superoxide at pH 7.8. a indicates water-soluble. UD=unable to determine due to interference with SOD assay. CAT: catalase activity measured by Clarke electrode. TBARS: Lipid peroxidation assay.

Structures and antioxidant activities of metalloporphyrins. Because metalloporphyrin catalytic antioxidants are catalytic, and not merely free radical scavengers, these compounds are much more potent antioxidants than dietary additives such as vitamin E that act stoichiometrically. The manganese meso-porphyrin catalytic antioxidants (see for example, FIG. 1) combine the broad spectrum of reactivity towards reactive species like the stoichiometric antioxidants with the catalytic efficiency of the endogenous antioxidant enzymes. Additionally, these synthetic compounds can be chemically modified to increase their ability to cross the blood brain barrier (BBB), as well as their availability to various subcellular compartments. Metalloporphyrins have plasma half lives that range from 4 to 48 hours. Most metalloporphyrins are not extensively metabolized by the body and are largely excreted unchanged in the urine. A previous limitation of the metalloporphyrin class of compounds has been the poor oral bioavailability. A major advancement in the field of catalytic antioxidants was the demonstration that AEOL11207, a lipophilic metalloporphyrin, protected against 1-methyl-4-phenyl-1,2,3,6-tetrahydropyridine (MPTP) neurotoxicity in vivo following oral administration, previously identified. This compound belongs to a new class of metalloporphyrins, the AEOL112 series of glyoxylate metalloporphyrins, which were designed to have greater lipid solubility, oral bioavailability, and cross the BBB. Several compounds in the AEOL 112-series have been shown to have good oral bioavailability and longer plasma half lives which should make them better candidates for treating chronic diseases.

Safety profile: A prototypical metalloporphyrin (AEOL10150; FIG. 1) has completed phase 1 trials in amyotrophic lateral sclerosis patients. Extensive safety studies of this compound have been completed in mice, monkeys and rats. No serious adverse events have been found. Safety studies for the structurally related compound, AEOL11207 need to be completed once its efficacy is established in animal studies. Of note, AEOL11207 was found to be negative in two mutagenicity tests (Ames' test and mouse lymphoma test). Serious adverse effects are not anticipated due to its structural similarity to AEOL10150.

### Example 2

### AEOL11207 Attenuates Seizures in a Mouse Model of Acute Mitochondrial Dysfunction

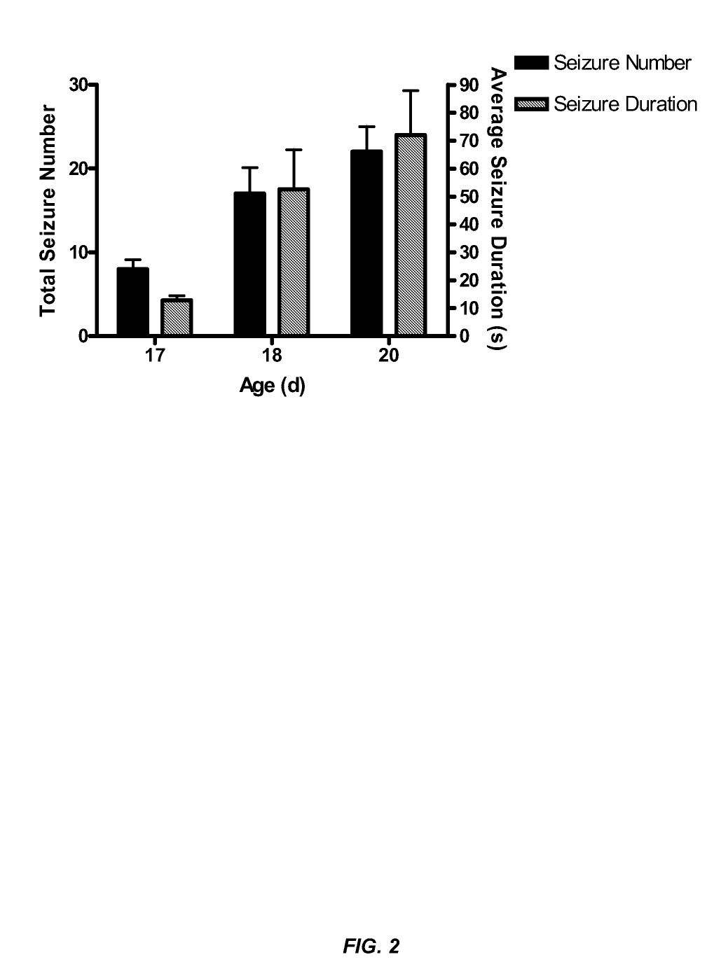

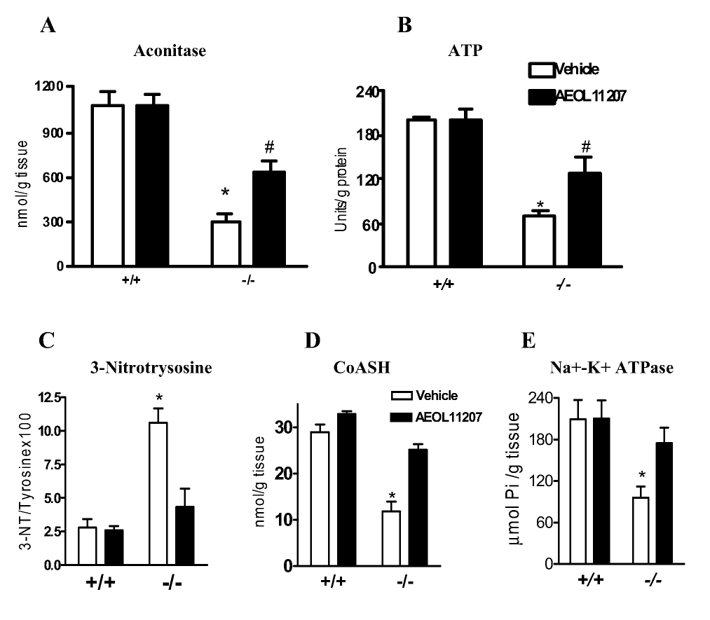

In anther exemplary experiment, AEOL11207 was found to attenuate seizures in a mouse model of acute mitochondrial dysfunction. The model utlizes cross-bred C57BL6XDBA2F2 (B6D2) mutant mice lacking manganese superoxide dismutase (MnSOD or Sod2), a critical mitochondrial antioxidant. In the second week of postnatal life (P14-P21) B6D2 Sod2−/− mice exhibit frequent episodes of spontaneous tonic-clonic seizures (see for example, FIG. 2), allowing their use as a model of epilepsy associated with mitochondrial disease for testing therapeutic interventions. When administered via s.c. route on postnatal day 5, AEOL11207 significantly attenuated behavioral seizure characteristics of Sod2−/− mice during the second to third week of post-natal life (see FIG. 5).

Previous studies have anecdotally reported the occurrence of spontaneous seizures in Sod2−/− mice during the second week of postnatal life, but their characteristics have not been identified or quantified. Here, the quantification of the averaged spontaneous seizure number and duration by postnatal day using daily video monitoring and seizure scoring of Sod2−/− mice is reported during the second week of postnatal life. FIG. 2 represents an exemplary histogram of the number and duration of seizures by postnatal day using daily video monitoring and seizure scoring of Sod2−/− mice is reported during the second week of postnatal life. Bars represent the mean seizure number or duration for each postnatal day. (n=3-5 mice per day).

### Example 3

### AEOL11207 Efficiently Penetrates the Blood-Brain Barrier (BBB) and Brain Mitochondria

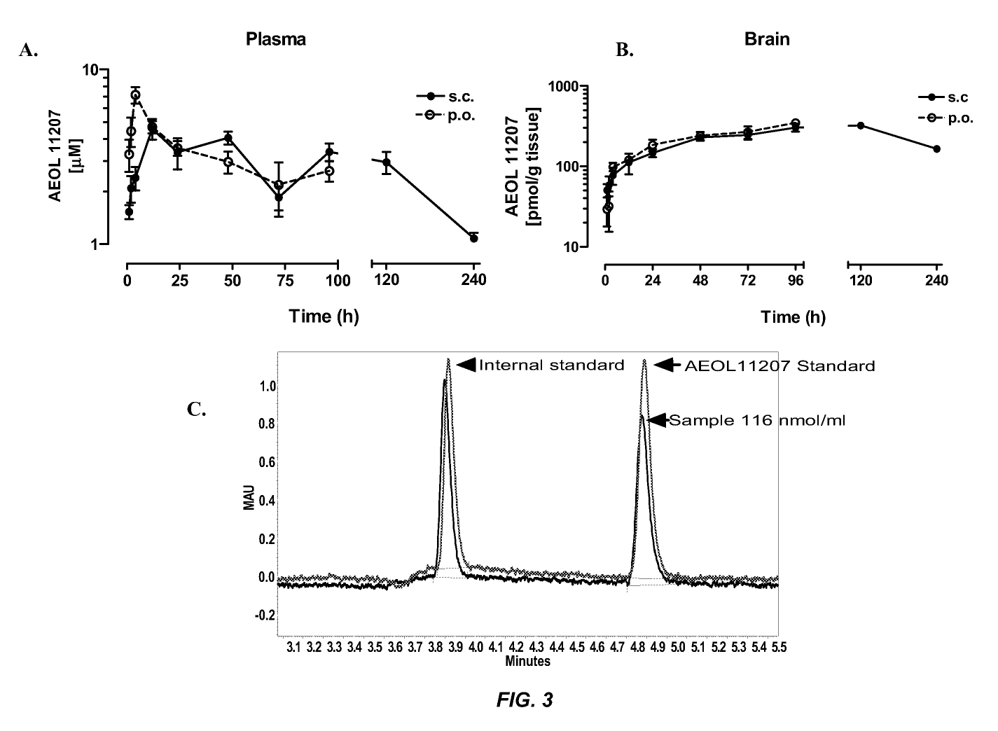

In another exemplary method, oral administration of AEOL11207 was examined for efficiently to penetrate the blood-brain barrier (BBB) and brain mitochondria. To determine if AEOL11207 penetrates the BBB, preliminary studies were conducted in mice following injection of AEOL 11207. Mice were given a single dose of the compound (15 mg/kg, p.o. AEOL 11207) or vehicle (control). At various times after injection, mice were perfused and brains (cortex) and plasma were extracted with methanol and samples analyzed by an HPLC method as described previously. The estimated concentration of AEOL 11207 in the brain following this dose and the extraction efficiency is within the protective range of this compound (˜30-100 nM) based on a paraquat (PQ2+)-induced cell injury assay (now shown). FIGS. 3A and 3B illustrate plasma and brain concentrations of AEOL11207 following s.c. or p.o. administration. FIG. 3B illustrates recovery of AEOL11207 from mitochondrial fractions of mice administered AEOL11207 via the s.c. route. Together, these results demonstrate the ability of AEOL11207 to cross the BBB following oral administration and penetrate brain mitochondria.

FIGS. 3A-3B illustrate exemplary concentrations of AEOL11207 in the plasma (3A) and brains (3B) of the C57BL/6 mice at different times points after a single dose of AEOL11207 (15 mg/kg) administered by the s.c or p.o. route. Points represent mean+S.E.M. Each point is the average of 3-4 animals. FIG. 3B illustrates recovery of AEOL11207 from mitochondrial fractions of mice administered AEOL11207 via the s.c. route. FIG. 3C illustrates an exemplary chromatogram of AEOL 11207 levels measured by HPLC with UV detection at 450 nm in mitochondrial fractions of mouse forebrain 24 hr after AEOL11207 15 mg/kg s.c. as previously described. Recovery of AEOL11207 from mitochondrial samples was determined to be ˜98%. Concentration of the standard is 120 nmol/ml and sample is 12 pmol/mg prot). X axis denotes response (nA) and Y axis denotes Time (min). The estimated concentration of AEOL 11207 in the brain following this dose and the extraction efficiency is within the protective range of this compound (˜30-100 nM) based on a paraquat (PQ2+)-induced cell injury assay. Oral administration of AE011207 attenuates oxidative damage and mitochondrial dysfunction in Sod2−/− mice.

In cell-free systems and isolated mitochondria, AEOL 11207 catalytically scavenges mitochondrial O2-, H2O2 and lipid peroxides decreasing the potential for oxidative stress induced damage to mitochondria and other cellular components. To determine whether AEOL11207 decreases mitochondrial dysfunction and corrects key bioenergetic parameters in Sod2−/− mice, activity was measured of the oxidant sensitive mitochondrial enzyme, aconitase and oxidant insensitive control, fumarase, ATP, 3-nitrotyrosine (3NT), a marker of oxidative damage to proteins, reduced coenzyme A (CoASH) which assesses the mitochondrial redox state and the activity of the sodium potassium ATPase (Na+-K+ ATPase). AEOL11207 significantly attenuated the decreases in aconitase activity, ATP levels, increases in 3NT levels, decreased CoASH levels and decreased Na+-K+ATPase activity observed in Sod2−/− mice confirming its ability to target oxidative damage, mitochondrial dysfunction and neuronal excitability. FIGS. 5A-5EC illustrate attenuation of aconitase inactivation (5A), ATP depletion (5B), 3NT formation (4C), CoASH depletion (5D) and decreased Na+-K+ ATPase activity (5E) in Sod2−/− mice by AEOL11207 (5 mg/kg, s.c. beginning PND5, n=*p<0.05, #p<0.01).

These exemplary experiments demonstrate for example, that 1) B6D2 Sod2−/− mice provide a model of mitochondrial dysfunction and epilepsy; 2) delayed systemic administration of a novel lipophilic metalloporphyrin ameliorates mitochondrial dysfunction, oxidative stress and seizure parameters in Sod2−/− mice; and 3) whole animal studies illustrate that AEOL11207 penetrates the BBB and accumulates in the CNS mitochondria. AEOL11207 is orally active, penetrates the BBB and brain mitochondria and protects against mitochondrial dysfunction in Sod2−/− mice. Thus, these data provide a compelling rationale for therapeutic development of this class of compounds.

Another exemplary study demonstrating the efficacy of AEOL11207 in a rat model of temporal lobe epilepsy was conducted. The rat model of temporal lobe epilepsy is initiated by a single injection of a chemoconvulasant agent which results in spontaneous epileptic seizure arise several days to weeks thereafter. Groups of rats administered vehicle, kainate (a chemoconvulsant used to initiate injury), kainate+AEOL11207 and AEOL11207 were monitored 6 weeks for behavioral seizures and indices of oxidative stress (GSH/GSSG, CoASH/CoASSG). AEOL11207 was administered at an arbitrary dose of 5 mg/kg, s.c. daily beginning 6 hr after injection of kainate (11 mg/kg). All of the rats in both groups experienced SE after kainate injection, and there was no difference in any of the characteristics of SE between the groups. Chronic seizures in animals are monitored by video recording (Q-See QD14B, Anaheim, Calif.) for 8 hours a day, 6 days/week in custom designed observation cages by a blinded observer. The time to develop chronic epilepsy (latency to chronic epilepsy), spontaneous seizure frequency, severity and duration was determined. As illustrated in Table 1, only one-third of rats treated with AEOL11207 developed chronic epilepsy in comparison with two-thirds in the kainate group during 6 weeks.

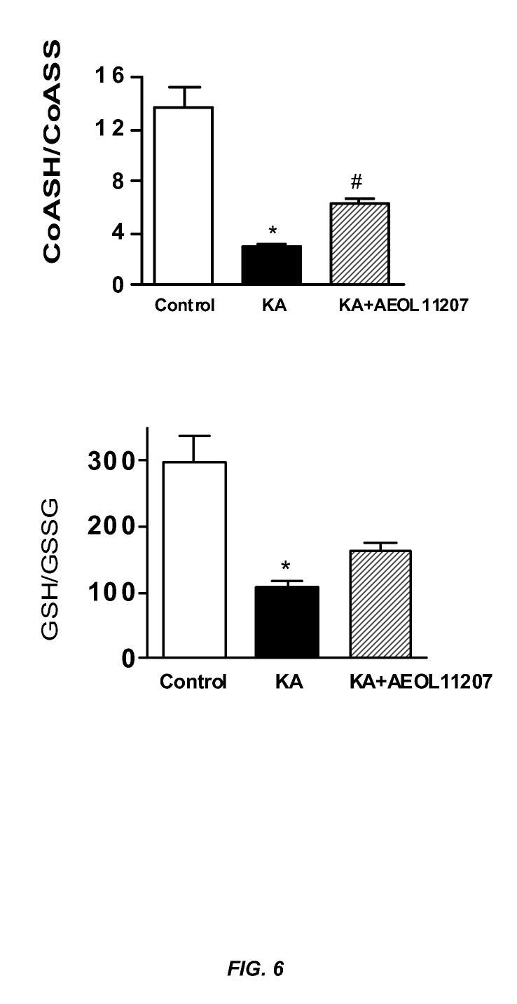

FIG. 6. illustrates that AEOL11207 decreases the frequency (6A) and total number (6B) of behavioral seizures in Sod2−/− mice. The data presented here illustrates that daily subcutaneous injections of AEOL11207 (5 mg/kg) to Sod2−/− mice beginning on P5 significantly decreased frequency of behavioral seizures during the second week of life (*p<0.05).

Materials and Methods: Sod mutant mice: Sod2−/− mice of a B6D2F2 background are obtained from B6D2F1 Sod2−/+ mice generated by crossing B6 Sod2−/+ males and D2 wild type female mice. Crossing B6D2F1 Sod2−/+ males and females yields ˜50% Sod2−/+ and 25% each Sod2+/+ and Sod2−/− mice. Each litter usually yields 2-3 Sod2−/− mice therefore, to achieve n=3 per timepoint, 10 litters (5 timepoints×2 routes) will be used which require ˜5-10 Sod2−/+ males and 20 females (25-40 mice F1 mice plus ˜20 wildtype D2 mice to start the colony). In total, using ˜45-60 mice will yield sufficient litters. Animals will be monitored on a daily basis to get an accurate birth date (P0). Pups will be genotyped after completion of analysis (Aim 1) or at PND5 (Aim 2) with tail DNA obtained by a 30 min proteinase K digestion followed by multiplex PCR amplification and agarose gel electrophoresis.

AEOL11207 measurement: AEOL11207 will be measured in plasma and brain samples of Sod2−/− and Sod2+/+ mice by HPLC methods as previously described for AEOL11207. Plasma drug levels will be measured in the adult mice (mothers) to confirm drug penetration via the placenta or milk. For comparison, AEOL11207 will also be measured in the plasma and brain of Sod2−/− mice injected with the drug via s.c. route at a dose of 5mg/kg on PND5. The drug levels will be measured in the latter group on PND 6, 7, 14 and 21.

Analysis: Data will be analyzed by for example, PKAnalyst (MicroMath software).

This study will confirm the BBB permeability and oral bioavailability of AEOL11207. The study will determine whether the compound crosses the placental barrier when administered during the gestation period by measuring drug levels on PND1 and if it passes through the mother's milk.

Based on the lipophillicity of AEOL11207 and previous data that shows efficient BBB permeability and accumulation the compound is expected to cross the placental and accumulate in breast milk. Comparison between the two routes of drug administration in the adult mice i.e. s.c. and diet will allow us to assess any variation in drug levels due to food intake. The goal of the oral or s.c. delivery via the mother receiving twice the dose (10 mg/kg) will be to achieve drug levels that are comparable to the pups receiving 5 mg/kg s.c.

Alternate strategies: 1) If toxicity is observed in the pups with the chosen dose with chronic dosing regimen, the dose (5 mg/kg) and/or the frequency of dosing can be reduced to once every 2-3 days. The pharmacokinetic analysis of AEOL11207 will reveal the optimal time to begin dosing to obtain sufficient brain concentrations.

Sod2+/+ and Sod2−/− mice treated with AEOL11207 via s.c. or diet will be analyzed for 1) mitochondrial oxidative stress/dysfunction (3NT, ATP, Na+-K+ ATPase, CoASH and aconitase/fumarase) and 2) seizure parameters (seizure frequency, interseizure interval and seizure duration via 24 hour video analysis). For comparison, mitochondrial dysfunction and seizures was also be assessed in the plasma and brain of Sod2−/− mice injected with the drug via s.c. route at a dose of 5mg/kg on PND5.

Mouse numbers: 1) Biochemical analysis: The following time-points will be selected for measurement of mitochondrial indices/oxidative stress: PND 3, 7, 21. Individual mice will be required for each index and timepoint due to the limited amount of tissue obtained from the neonatal mouse. The number of mice anticipated for this aim was: n=4-6 mice per end-point×3 end-points and 2 genotypes=36 Sod2+/+ and 36 Sod2−/−. To obtain 36 Sod2−/− mice, we anticipated using ˜12 litters from Sod2−/+ mice. 2) Seizure parameters: Approximately 20-30 mice of each genotype (Sod2+/+ and Sod2−/−) are anticipated for these studies which will require ˜6 litters.

Methods:3-nitrotyrosine (3-NT) measurement by HPLC 3-NT assay are performed with HPLC equipped electrochemical detector using methods as previously described in the literature. The potentials of the electrochemical detector are set at 180/240/350/500/600/670/810/830 mV. Analyte separation is conducted on a TOSOHAAS (Montgomeryville, Pa.) reverse-phase ODS 80-TM C-18 analytical column (4.6 mm×250 cm; 5 pm particle size). A two-component gradient elution system was used with component A of the mobile phase composed of 50 mM sodium acetate pH 3.2, and component B composed of 50mM sodium acetate and 40% methanol. The following gradient elution profile was used: 0-25 min, 100% A; 25-35 min, linear ramp to 50% B; 35-40 min, isocratic 50% B; 40-45 min, linear ramp to 100% A; 45-50 min, isocratic 100% A at a flow rate of 0.6 ml/min. 3-NT was expressed as a ratio of 3-NT to tyrosine.

Measurement of AMP, ADP and ATP: The levels of AMP, ADP and ATP are quantified by HPLC-UV set at 258 nm following previous described. Analyte are separated by 5 μM, 4.6×250 cm C-18 reversed-phase column. Mobile phase is composed of 50 mM KH2PO4, 10% methanol, 3 mM TBAS and adjusted to pH 6.0 and flow rate set at 0.8 ml/min.

Aconitase and fumarase activity assay: Aconitase and fumarase activity are measured in mitochondrial fraction which isolated as previously described. Briefly, brain tissues are homogenized with a Dounce tissue grinder (Wheaton, Millville, N.J.) in ice-cold mitochondrial isolation buffer (70 mM sucrose, 210 mM mannitol, 5 mM Tris HCl, 1 mM EDTA pH 7.4). After homogenization, the suspensions are centrifuged at 800 g for 10 minutes at 4° C., and the supernatants centrifuged at 17,000 g for 10 minutes at 4° C. The pellets are washed by mitochondrial isolation buffer and centrifuged at 17,000 g for 10 minutes at 4° C. again. Aconitase activity is measured spectrophotometrically following previous described by monitoring the formation of cis-aconitate from isocitrate at 240 nm in 50 mM Tris HCl, pH 7.4, containing 0.6 mM MnCl2 at 25° C. Fumarase activity is measured by monitoring the increase in absorbance at 240 nm at 25° C. in a 1 ml reaction mixture containing 30 mM potassium phosphate, pH 7.4, 0.1 mM L-malate.

Chronic Video Monitoring and Seizure Scoring: Sod2−/− mice and wild type littermates aged P7-˜P21 are digitally recorded 8 hrs/day in a custom designed plexiglass cage using a Q-See QD14B video monitoring system connected to a recordable DVD. DVD recordings are saved and reviewed by a trained observer blind to genotype and/or treatment for scoring of seizure severity, duration, and frequency. Seizure severity will be rated using the following scale: 1=immobilization & staring, 2=head-nodding, occasional forelimb clonic and tonic activity, 3=continuous forelimb clonic and tonic activity, 4=generalized seizures with falling, running and jumping.

Statistical analysis: Two-way ANOVA will be used to determine the differences between treatment and genotype. For differences between seizure parameters, one-way ANOVA with Neuman-Keul post-hoc analysis will be used. Group measures are expressed as mean±SEM. The statistical significance of differences are assessed with the Students t-test. The level of significance will be set at p<0.05.

Toxicity of chronically administered metalloporphyrin: Metalloporphyrins used here have manganese as the metal center for catalyzing redox reactions. One possibility is that its chronic presence may result in the release of manganese from porphyrin rings and a manganese based neurotoxicity. This scenario is unlikely for the following reasons: 1) manganic porphyrins are extremely stable, they have been found to keep the manganese chelated even in the presence of millimolar amounts of EDTA; 2) several of these compounds have been found to be safe and efficacious when used in both in vitro and in vivo models of neurodegeneration; and 3) several additional glyoxylate metalloporphyrins are available to serve as back-up compounds to overcome any issues with AEOL 11207 (e.g. AEOL11209, FIG. 1).

### Example 4

### AE011207 Attenuates Oxidative Damage and Mitochondrial Dysfunction in Sod2−/− Mice

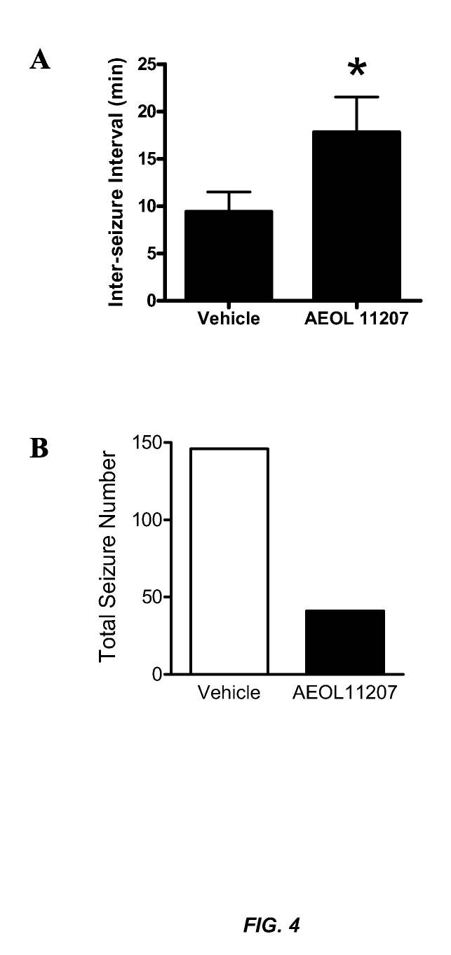

In another exemplary method, experimental studies were performed to determine whether pharmacological removal of ROS with AEOL11207 inhibited epilepsy resulting specifically from mitochondrial oxidative stress in mutant mice deficient in MnSOD or Sod2, a critical mitochondrial antioxidant (see FIGS. 4 and 5). AEOL11207 is an orally active metalloporphyrin catalytic antioxidant that penetrates the blood-brain barrier and brain mitochondria. In cell-free systems and isolated mitochondria, AEOL 11207 catalytically scavenges mitochondrial O2-, H2O2 and lipid peroxides decreasing the potential for oxidative stress induced damage to mitochondria and other cellular components. Sod2−/− mice were used in a mixed background (e.g. B6D2F2) which live ˜2-3 weeks postnatally and develop epilepsy during the 2nd week of life. Therefore, these longer-lived Sod2−/−(B6D2) mice provide a model of epilepsy associated with mitochondrial disease in which therapeutic interventions can be tested. To determine whether AEOL11207 decreases mitochondrial dysfunction and corrects key bioenergetics parameters, the activity of the oxidant sensitive mitochondrial enzyme, aconitase and oxidant insensitive control, fumarase; ATP, Na+-K+ ATPase activity, and a marker of oxidative stress, 3-nitrotyrosine (3NT) were assessed. AEOL11207 significantly attenuated the decreases in aconitase activity (not shown), ATP levels, Na+-K+ ATPase activity and 3NT formation observed in Sod2−/− mice in support of its ability to target oxidative damage and mitochondrial dysfunction (FIG. 5). The data demonstrate that systemic administration of AEOL11207 ameliorates mitochondrial dysfunction, oxidative stress and seizure parameters in Sod2−/− mice (FIG. 5). Together these data provide a compelling rationale for therapeutic development of this class of compounds.

FIG. 6 are histograms representing attenuation of frequency of behavioral seizures, 3NT formation, Na+-K+ ATPase activity, and CoASH levels in B6D2F2 Sod2−/− or +/+ littermates after treatment with AEOL11207 (5 mg/kg, s.c.) beginning day 5, n=4-16*p<0.05. FIG. 5. is a chromatogram representing AEOL 11207 levels measured by HPLC-UV at 450 nm in mitochondrial fractions of mouse forebrain 24 hr after AEOL11207 15 mg/kg s.c. as previously described. Recovery of AEOL11207 from mitochondrial samples was ˜98%. Concentration of the standard is 120 nmol/ml and sample is 12pmol/mg prot).

### Example 5

### AEOL11207 Inhibits Acquired Epilepsy Development in Adult Animals i.e. Epileptogenesis

In another exemplary experiment, pharmacological removal of ROS was examined for its inhibition on kainate-induced epileptogenesis. A pilot was performed in which groups of rats administered vehicle (n=4), kainate (n=6), kainate+AEOL11207(n=6) and AEOL11207 (n=4) were monitored 6 weeks for behavioral seizures (Table 1) and indices of oxidative stress (GSH/GSSG, CoASH/CoASSG) (FIG. 6).

AEOL11207 was administered at a dose of 5 mg/kg, s.c. daily beginning 6 hr after injection of kainate (1 mg/kg). All of the rats in both groups experienced SE after kainate injection, and there was no difference in any of the characteristics of SE between the groups. Chronic seizures in animals were monitored by video recording (Q-See QD14B, Anaheim, Calif.) for 8 hours a day, 6 days/week in custom designed observation cages by a blinded observer. The time to develop chronic epilepsy (latency to chronic epilepsy), spontaneous seizure frequency, severity and duration was determined as previously described. As shown in Table 1, only one-third of rats treated with AEOL11207 developed chronic epilepsy in comparison with two-thirds in the kainate group during 6 weeks. In the remaining 2 rats in the kainate+AEOL11207 group that developed epilepsy, seizure duration was decreased by ˜15% (Table 1, FIG. 6) but no changes were observed in the number of seizures per animal. None of the rats in the control or AEOL11207 alone groups developed seizures. Moreover, CoASH/CoASSG and GSH/GSSG ratios were significantly improved in AEOL11207-treated rats compared to controls (FIG. 6). This pilot study suggests a potential antiepileptogenic effect of AEOL11207 underscoring the importance of further detailed studies. FIG. 6 and Table 1 illustrate the effect of AEOL11207 on kainate-induced chronic epilepsy development (n=6/group) and oxidative stress (n=4-6 per group).

TABLE 1

| | Kainate | Kainate + AEOL11207 |
|---|---|---|
| # Rats with spontaneous seizures | 4/6 (66.7%) | 2/6 (33.3%) |
| Duration of seizures (sec.) | 33.3 ± 1.9 | 28.3 ± 2.2*p < 0.05 |

### Example 6

### Treatment Of Epilepsy, Mitochondrial Dysfunction and Neuronal Injury in Sod2−/− Mice with a Lipophilic Metalloporphyrin

Epileptic seizures are a common feature observed in children with inherited mitochondrial diseases. The objective of this study was to determine if a novel lipophilic metalloporphyrin antioxidant modulates behavioral seizures, mitochondrial dysfunction, and neuronal injury in a mouse model of mitochondrial dysfunction and epilepsy. The animal model utilizes cross-bred C57BL6XDBA2F2 (B6D2F2) mutant mice lacking manganese superoxide dismutase (MnSOD or Sod2), a critical mitochondrial antioxidant. In the second to third week of postnatal life (P14-P21) B6D2F2 Sod2−/− mice exhibited frequent episodes of spontaneous tonic-clonic seizures, providing a model of epilepsy associated with mitochondrial disease. A newly developed glyoxylate series of metalloporphyrins shows a high potency for catalytic removal of endogenously generated reactive oxygen species in respiring brain mitochondria. The effect of a potent lipophilic metalloporphyrin in this series, AEOL11207, was determined on video recorded behavioral seizure characteristics (seizure number, frequency, duration, and severity) of Sod2−/− mice during the second to third week of post-natal life. Sod2−/− mice treated with AEOL11207 showed a decrease in the total number and frequency of behavioral seizures but not seizure duration or severity, and a significant increase in average lifespan compared to controls (14.01±3.95 days to 20.33±2.00 days). Indices of mitochondrial oxidative damage (aconitase inactivation, 3-nitrotyrosine formation), depletion of cellular antioxidants or their building blocks (glutathione, cysteine and ascorbate), and bioenergetic targets controlling neuronal excitability (ATP, Na+-K+ ATPase activity and astrocytic glutamate transporter; Glt-1 levels) were significantly attenuated in the brains of AEOL11207-treated Sod2−/− mice compared to vehicle-treated Sod2−/− mice. These results demonstrate the ability of a synthetic lipophilic metalloporphyrin to attenuate behavioral seizures, mitochondrial dysfunction and bioenergetic parameters known to alter neuronal excitability in a mouse model of mitochondrial disease and epilepsy. The data suggest that mitochondrial oxidative stress may be a novel therapeutic target for the treatment of epilepsies associated with mitochondrial diseases.

Epileptic seizures are the most common clinical feature in children with inherited mitochondrial diseases. General and partial seizures with mitochondrial encephalopathy can be caused by mitochondrial dysfunction arising from mitochondrial mtDNA mutations (Shoffner, et al. (1990) Cell 61, 931-7; Wallace, et al. (1988) Cell 55, 601-10). It has been suggested that mitochondrial dysfunction may be an important biochemical trigger of epileptic seizures (Kunz, W. S. (2002) Curr Opin Neurol 15, 179-84; Patel, M. (2004) Free Radic Biol Med 37, 1951-62). Results from this and other laboratories suggests that mitochondrial oxidative stress and resultant dysfunction can render the brain more susceptible to epileptic seizures (Liang, et al. (2000) Neuroscience 101, 563-70; Liang & Patel, (2004) Free Radic Biol Med 36, 542-54; Kudin, et al. (2002) Eur J Neurosci 15, 1105-14). Mitochondria have several important functions that include cellular ATP production, control of apoptotic/necrotic cell death, reactive oxygen species (ROS) formation and neurotransmitter biosynthesis. Which of these critical mitochondrial functions contributes to increased seizure susceptibility associated with mitochondrial diseases remains unknown. Additionally, mitochondrial dysfunction is a consequence of various neurological insults such as neonatal hypoxia and trauma, which are known risk factors for childhood seizures indicating that mitochondrial dysfunction per se may be a common pathway contributing to epileptogenesis. Advances in understanding the molecular and cellular biology of mitochondrial (dys)function may lead to novel approaches for the prevention and treatment of neurological disorders, including childhood epilepsies.

To understand the basis of epilepsy in mitochondrial diseases it is useful to develop animal models in which epileptic seizures arise due to mitochondrial dysfunction, thus providing a valuable tool to assess potential therapies for catastrophic childhood epilepsies associated with mitochondrial diseases. Mutant mice lacking manganese superoxide dismutase (MnSOD or Sod2), a critical mitochondrial antioxidant, provide such a model. Mitochondrial disease has been characterized in Sod2 deficient mice generated in several background strains. Whereas Sod2^{-/-} mice bred from a C57B6 background (B6 Sod2^{-/-}) are embryonic lethal, CD-1 Sod2^{-/-} mice develop and live approximately 8-10 days postnatal (Melov, et al. (1999) Proc Natl Acad Sci USA 96, 846-51). Recently, first generation Sod2^{-/-} mutant mice (B6D2F1) from a mixed background (C57BL/6JX DBA/2J) have been generated, which live approximately 3 weeks without pharmacological intervention (Lynn, et al. (2005) Free Radic Biol Med 38, 817-28). In the second-third week of postnatal life (P14-P21) B6D2F1 and B6D2F2 Sod2^{-/-} mice exhibit frequent episodes of spontaneous tonic-clonic seizures (Lynn et al.,). Therefore, increased life-span of the cross-bred B6D2 Sod2^{-/-} mice provide a model of epilepsy associated with mitochondrial disease in which therapeutic interventions can be tested.

Metalloporphyrin catalytic antioxidants are small molecule mimics of superoxide dismutase and/or catalase, and also potent detoxifiers of lipid peroxides and peroxynitrite (reviewed in Day, B. J. (2004) Drug Discov Today 9, 557-66). Because they are catalytic, and not merely free radical scavengers, these compounds are much more potent antioxidants than dietary additives such as vitamin E that act stoichiometrically. The manganese mesoporphyrin catalytic antioxidants combine the broad spectrum of reactivity towards reactive species like the stoichiometric antioxidants with the catalytic efficiency of the endogenous antioxidant enzymes. Additionally, these synthetic compounds can be chemically modified to increase their ability to cross the blood brain barrier (BBB), as well as their availability to various subcellular compartments. A previous limitation of the metalloporphyrin class of compounds has been the poor BBB permeability. Treatment of short-lived Sod2−/− mice in the CD-1 background with manganese tetrakis 5, 10, 15, 20 . . . porphyrin (MnTBAP) ameliorated cardiomyopathy but not neurodegeneration (Melov et al.,) whereas EUK8 or EUK134 ameliorated spongiform encephalopathy and neurodegeneration (Melov et al., 1999). A major advancement in the field of catalytic antioxidants was the demonstration that AEOL11207, a lipophilic metalloporphyrin, protected against 1-methyl-4-phenyl-1,2,3,6-tetrahydropyridine (MPTP) neurotoxicity in vivo following oral administration (Melov, et al. (1999) Proc Natl Acad Sci USA 96, 846-51; Lynn, et al. (2005) Free Radic Biol Med 38, 817-28). This compound belongs to a new class of metalloporphyrins, the AEOL112 series of metalloporphyrins (Trova, et al. (2003) Bioorganic & Medicinal Chemistry 11, 2695-707), which were designed to have greater lipid solubility, oral bioavailability, and cross the BBB. The objective of this study was to characterize mitochondrial dysfunction and seizures in the B6D2F2 Sod2^{-/-} mouse model and determine the effect of AEOL11207, a lipophilic metalloporphyrin antioxidant resultant epilepsy and the ability of in the of acute mitochondrial dysfunction.

### RESULTS

AEOL11207 brain levels and antioxidant activity: To determine the bioavailability of AEOL11207 in neonatal mice, we measured the concentrations of AEOL11207 in mouse forebrain homogenates 24 h following treatment with a single dose of 5 mg/kg s.c. The concentration of AEOL11207 in the mouse forebrain was ˜30 nM and its recovery from the samples was determined to be ˜98%. To determine if AEOL11207 treatment resulted in increased antioxidant activity in brain mitochondria, we measured SOD2 activity in mitochondrial fractions from the forebrain of 15-16 day-old mice injected with vehicle or AEOL11207. Cyanide insensitive SOD activity, which is indicative of mitochondrial SOD activity in Sod2−/− mice receiving AEOL11207 treatment was ˜40% (0.62±.0.07 units/mg protein, mean±S.E.M, n=6) compared to Sod2+/+ mice (1.56±0.17 units/mg protein, mean±S.E.M n=6). There was no SOD2 protein band detectable by Western blot analyses in Sod2−/− mice regardless of treatment (data no shown).

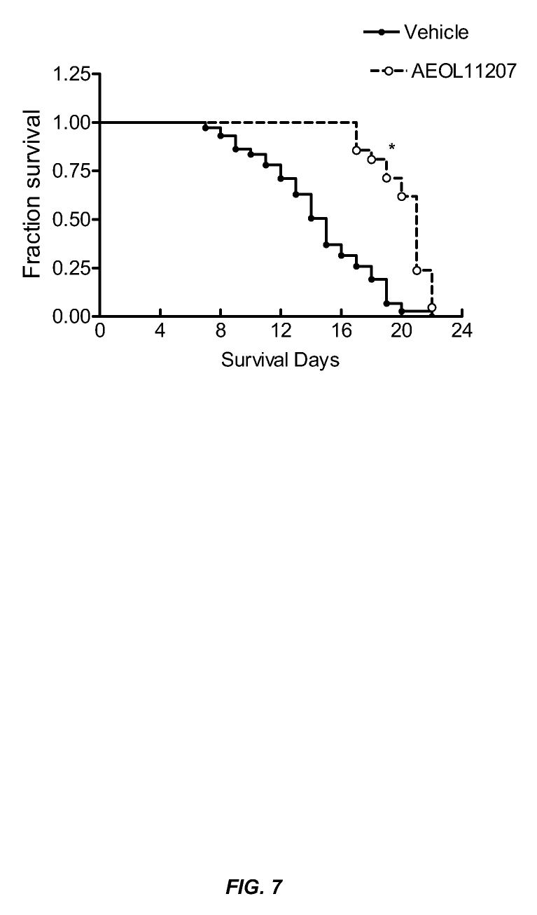

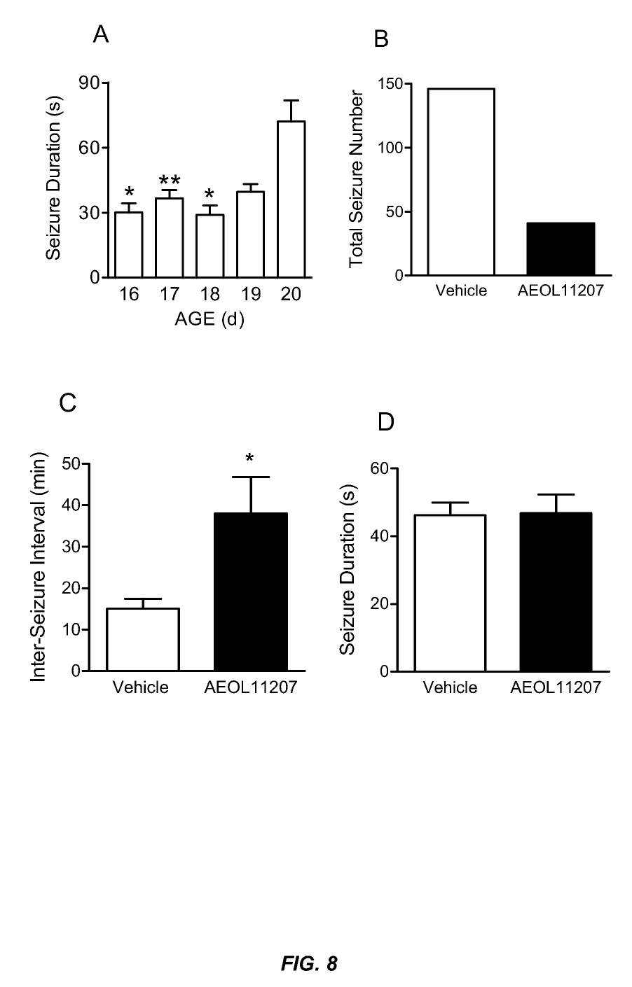

Lifespan and seizures B6D2F1 Sod2−/− mice had an average lifespan of 14.42±0.44 days (n=73) which is similar to previously reported results (Huang, et al. (2001) Free Radic Biol Med 31, 1101-1110). Sod2−/− mice treated daily with AEOL 11207 (5mg/kg) had a significant increase in average lifespan compared to vehicle treated Sod2−/− mice to 20.383±0.43 days (n=21). A prominent feature of the survival curves is that dramatically increased the percentage of Sod2 −/− mice living beyond 2 weeks of age with AEOL11207 treatment. Only ˜48% of vehicle-treated Sod2−/− mice survived beyond 15 days old, whereas, all of Sod2−/− mice treated with AEOL 11207 survived beyond 15 days old (FIG. 7). Daily AEOL11207 treatment (2.5 mg/kg) had no significant increase in average lifespan (data not shown). The averaged duration of spontaneous behavioral seizures from vehicle-treated Sod2−/− mice increased in a time-dependent manner from 16 to 20 days old with a significant increase present by postnatal day 20 (FIG. 8A). All vehicle-treated Sod2−/− mice exhibited frequent episodes of spontaneous tonic-clonic seizures beginning in the second week of postnatal life (P15-P20) (see supplemental video) compared to 37% of AEOL11207 treated Sod2−/− mice based on daily observation. AEOL11207 treated Sod2−/− mice showed a decrease in the total number and frequency of spontaneous seizures, but no significant changes in seizure duration or severity (FIG. 8B, C, D). Wild-type mice treated with AEOL11207 at the same dose showed no ill effects up to 20 days of treatment and no mortality.

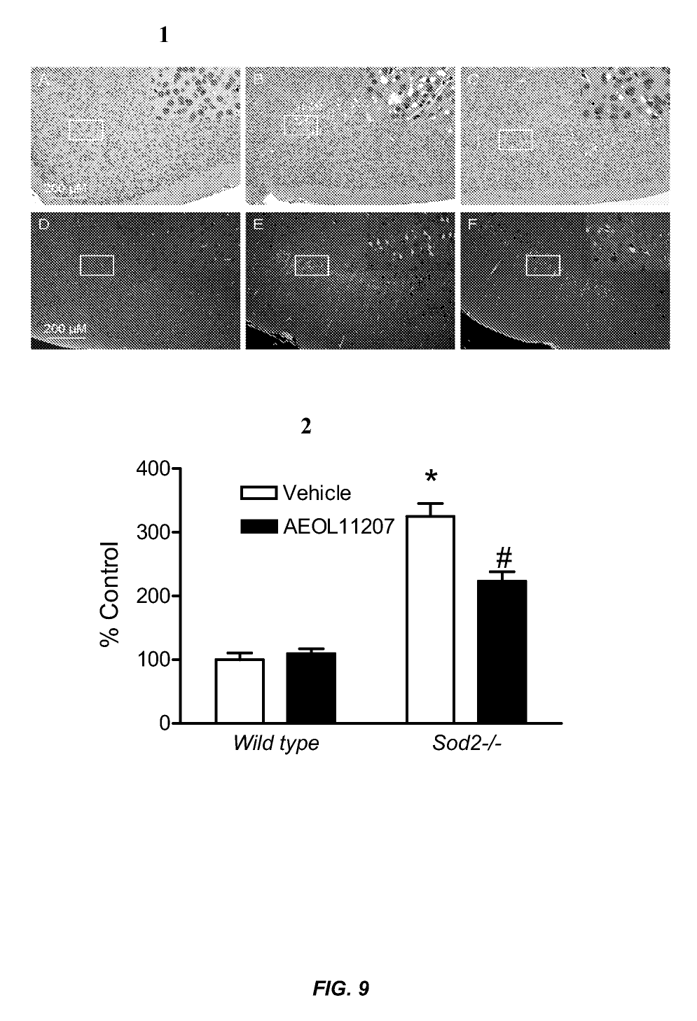

Histopathological analysis To determine the pathological damage related to the neurological symptoms, serial coronal section from brains of Sod2 −/− mice at P15-16 were examined by H&E and Fluoro-Jade B staining. No neuronal damage was observed in any brain region of wild type animals by H&E staining (FIG. 9 panel 1, A). In combination with the frequent episodes of spontaneous tonic-clonic seizures beyond the second week of postnatal life, Sod2 −/− mice bred from the B6D2F1 background developed “vacuolar degeneration” in regions of the cerebral cortex, predominately in the parietal cortex (FIG. 9 panel 1, B), although also in the frontal and piriform cortex, brainstem, thalamus, and in the pyramidal layer of hippocampus. Vacuoles ranged from 4 to 40 μm, imposed in neighboring structures such as neurons and blood vessels. These neuropathological results are consistent with those identified in patients with mitochondrial encephalopathy (spongiform encephalopathy) (Harper, et al. (Oxford University Press, New York), Vol. 10.) and also consistent with previous experimental observations (Lynn, et al. (2005) Free Radic Biol Med 38, 817-28; Melov, et al. (2001) J Neurosci 21, 8348-53). Histopathological damage including vacuole size and number were decreased by AEOL11207 treatment (FIG. 9 panel 1, C). To quantify the neuroprotective effects of AEOL11207 on histopathological damage, Fluoro-Jade B staining, a sensitive marker assessing degeneration of neuronal cell bodies and processes (Hopkins, et al. (2000) Brain Res 864, 69-80), was performed. It has been demonstrated that Fluoro-Jade B is a more sensitive, reliable and definitive marker of neuronal degeneration than silver staining techniques (Schmued, et al. (1997) Brain Res 751, 37-46; Schmued & Hopkins (2000) Brain Res 874, 123-30). No significant Fluoro-Jade B staining, indicative of neuronal degeneration, was observed in any brain region of control animals (FIG. 9 panel 1, D). However, significant staining (degeneration) was observed in the cell bodies and terminals in regions of the cerebral cortex, predominately in the parietal cortex (FIG. 9 panel 1, E). Significant protection of neuronal degeneration was observed in the AEOL11207 treatment group compared to vehicle treatment (FIG. 9 panel 1, 3F). The relative fluorescence density quantified by Image J increased ˜225% in the parietal cortex of Sod2 −/− mice compared to the control group and was significantly attenuated ˜50% by AEOL 11207 administration compared to vehicle treatment (FIG. 9 panel 2).

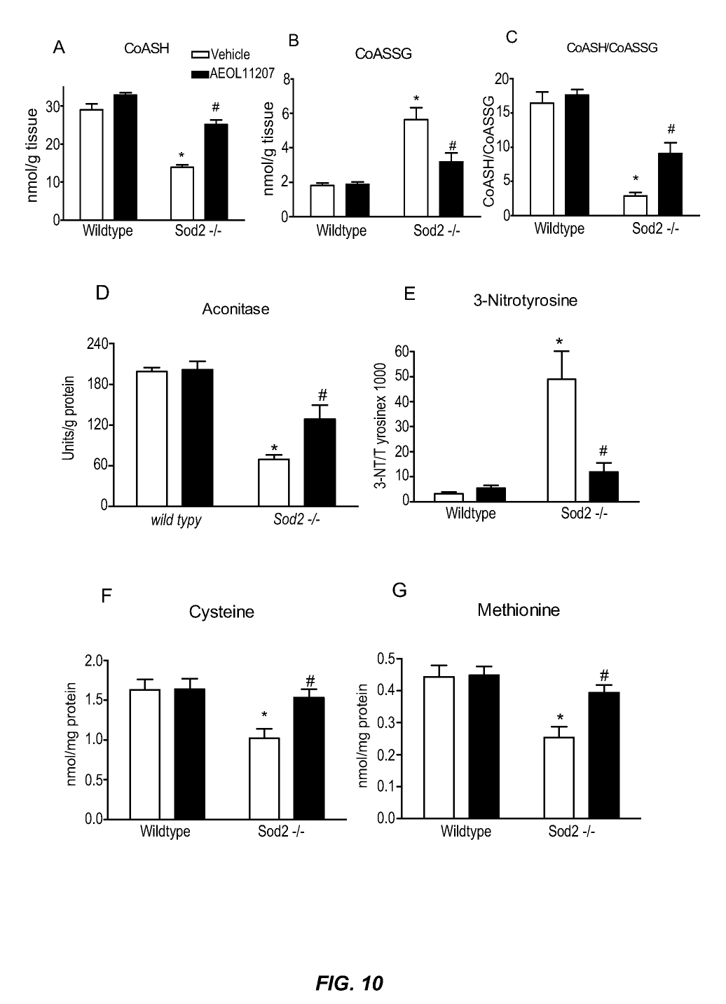

Mitochondrial oxidative stress production To determine if increased oxidative stress occurred in mitochondrial fractions, the levels of CoASH, CoASSG, aconitase, ascorbate, cysteine, methionine and 3-NT were examined in the forebrain of Sod2 −/− mice at 15-16 days old. GSH is the most abundant thiol containing antioxidant in tissues and brain (Meister & Anderson (1983) Annu Rev Biochem 52, 711-60) and plays an important role in preventing oxidative damage. Depletion of GSH has been demonstrated in many acute and chronic neuronal disorders (Sims, et al. (2004) J Bioenerg Biomembr 36, 329-33; Liu, et al. (2004) Ann NY Acad Sci 1019, 346-9; Perry, et al. (1982) Neurosci Lett 33, 305-10; Liang & Patel, (2006) Free Radic Biol Med 40, 316-22). CoASH and CoASSG are primarily compartmentalized within mitochondria and exchange thiol with GSH and GSSG, their measurement in intact tissue provides a reliable assessment for redox status in the mitochondria to overcome artifactual changes in GSH and GSSG associated with subcellular fractionation isolation (Liang & Patel, (2006) Free Radic Biol Med 40, 316-22; O'Donovan, et al. (2002) Pediatr Res 51, 346-53). The level of CoASH was depleted ˜50% and CoASSG was increased ˜210% resulting in a CoASH/CoASSG ratio that was reduced to 18% of control in the forebrain of Sod2 −/− mice (FIG. 10 A). The level of GSH was not changed in the forebrain cytosol fractions of Sod2 −/− mice (Data not shown). It has been suggested that the mitochondrial glutathione pool plays a far more important role in maintaining cell viability following toxic insults compared to the cytoplasmic pool (Meredith & Reed (1982) J Biol Chem 257, 3747-53). Aconitase has been reported to be highly sensitive to superoxide radical and peroxynitrite inactivation (Gardner, & Fridovich (1992) J Biol Chem 267, 8757-63; Patel, et al. (1996) Neuron 16, 345-55; Gardner, et al. (1997) J Biol Chem 272, 25071-6). The activity of aconitase in mitochondria was significantly reduced 65% compared to controls (FIG. 10B), which is consistent with previously reported results (Melov, et al. (1999) Proc Natl Acad Sci USA 96, 846-51). By contrast, the activity of aconitase in the cytosol and fumarase in the mitochondria showed no significant reduction in the forebrain of Sod2 −/− mice (data not shown). Both cysteine and methionine are sulfur-containing amino acids with antioxidant function and susceptible to oxidation by almost all of forms of ROS (Metayer, et al. (2008) J Nutr Biochem 19, 207-15). The levels of cysteine and methionine were reduced ˜38% and 43% in the forebrain mitochondrial fraction of Sod2 −/− mice compared with control (FIGS. 10C and D). 3-NT is an indicator of free nitro tyrosine residues in proteins following the reaction with nitrating oxidants. It has been demonstrated that peroxynitrite (ONOO⁻), a reaction product of NO and superoxide anion radical, is likely a primary source and major contributor to tyrosine nitration in physiological and pathological events in vivo (Sawa, et al. (2000) J Biol Chem 275, 32467-74). 3-NT formation by nitration of tyrosyl residues has been a well documented marker of OONO⁻ production both in vitro and in vivo (Beckman et al. (1994) Methods Enzymol 233, 229-40). The concentration of 3-NT was significantly increased 15 fold in forebrain mitochondrial fractions of Sod2 −/− mice compared with controls (FIG. 10E) suggesting that OONO⁻ production is markedly amplified in the absence of SOD2 which is likely due to increased superoxide radical production. To determine if direct scavenging of ONOO− could result in decreased 3NT levels by AEOL11207, we measured its ability to inhibit ONOO-induced oxidation of dihydrorhodamine-123. The IC50 of AEOL11207 was 3.7 μM which is ˜100 times greater than its measured brain concentration (˜30 nM) suggesting that its protective effects may not be due to scavenging ONOO- directly.

Ascorbate, an endogenous antioxidant, showed no alteration in the forebrain of Sod2 −/− mice (data not shown). To the best of our knowledge, the results concerning CoASH, CoASSG, cys, met and 3-NT have not previously been reported in Sod2 −/− mice. Administration of AEOL 11207 significantly attenuated MnSOD deficiency-induced decreases in aconitase, CoASH, cysteine, methionine, and increased CoASSG and 3-NT (FIGS. 10 A, B, C and D).

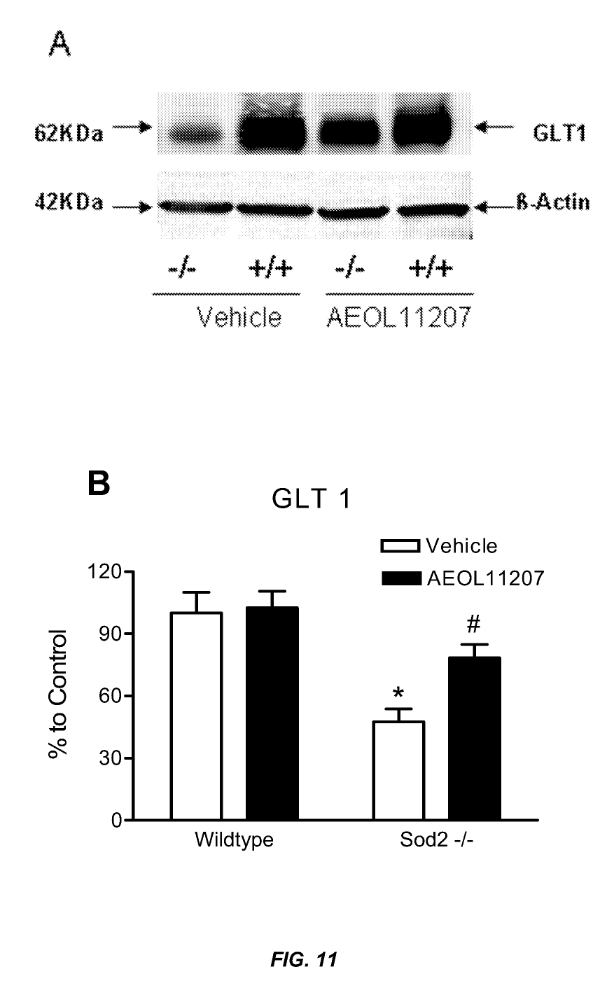

Glutamate transporter expression It has been demonstrated that astroglial glutamate transporters, EAAT2 (Glt-1) accounts for the majority high affinity glutamate uptake and therefore maintain synaptic cleft glutamate from reaching excitotoxicity (Suchak, et al. (2003) J Neurochem 84, 522-32). To determine the mechanism of mitochondrial dysfunction could increase seizure susceptibility, the expression of EAAT2 (Glt-1) in Sod2−/− mice was examined. In this study, the expression of glial transporter (GLT-1) was significant decreased more than 50% in the hippocampus of Sod2−/− mice compared to their controls. AEOL11207 treatment attenuated the decreased in GLT1 expression of Sod2−/− mice (FIG. 11).

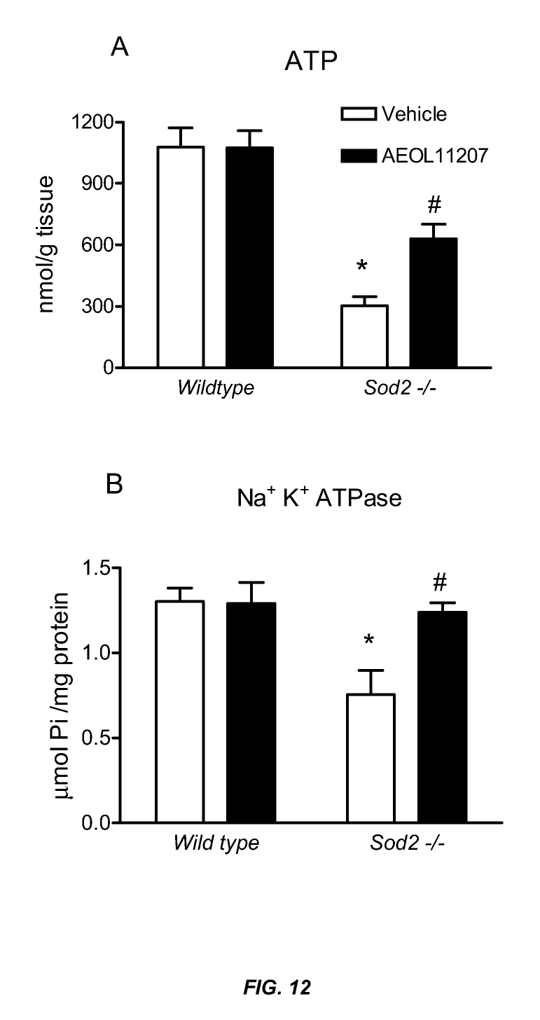

ATP production and Na⁺, K⁺ ATPase activity One of the major functions of mitochondrion is to synthesize ATP. The measurement of its production is a good process for the evaluation of mitochondrial function, especially in the brain where glycolysis provides much less ATP production than in other organs. The level of ATP was significantly reduced 70% in forebrain homogenates of Sod2−/− mice compared with controls. AEOL11207 administration attenuated 50% of the depletion of ATP in forebrain homogenates of Sod2−/− mice (FIG. 12A). Na⁺K⁺-ATPase (EC 3.6.3.9) is a membrane enzyme that maintains neuronal membrane potential through the active transport of sodium and potassium ions to regulate neuronal excitability. To further determine the activity of the enzyme under conditions of ATP depletion and increased oxidative stress, its activity was assessed. The level of Na⁺ K⁺-ATPase significantly decreased 42% in forebrain tissue homogenates of Sod2−/− mice compared to controls. AEOL11207 administration significantly restored enzyme activity (FIG. 12B).

DISCUSSION: In this study we have characterized a mouse model of mitochondrial dysfunction in which epileptic seizures are prominent and attenuation of seizures and mitochondrial dysfunction by a lipophilic metalloporphyrin catalytic antioxidant. In this work, Sod2 −/− mice provide a model to study that the mechanism(s) of mitochondrial dysfunction increases susceptibility to epilepsy. A catalytic antioxidant, metalloporphyrin AEOL11207 which prevents against oxidative stress resultant mitochondrial dysfunction significantly attenuates total numbers frequency and duration of seizures.

In this study, our result revealed a significant decrease in aconitase activity in forebrain mitochondrial fractions of Sod2 −/− mice. The substantial loss of complexes activity resultant an impaired electron flux through the electron transport chain (ETC) causes a decrease in ATP synthesis. Moreover, aconitase is one of the most important components in tricarboxylic acid cycle which provide reduced NADH and FADH for ETC to synthesize ATP. The significant ATP depletion in forebrain of Sod2−/− mice may be due to both diminished complexes and aconitase activity.

Our previous work has found that the expression of glial (GLT-1 and GLAST), but not one neuronal (EAAC-1) transporter was decreased in aged Sod2−/+ mice compared to their age-matched wild-type controls (Liang & Patel, (2004) Free Radic Biol Med 36, 542-54). Decreased expression of GLT-1 and GLAST has been also observed in the cortex of rats with genetic absence epilepsy (Dutuit, et al. (2002) J Neurochem 80, 1029-38) and in the hippocampus of epileptic EL mice (Ingram, et al. (2001) J Neurochem 79, 564-75). It is strongly indicated dysfunction of glutamate transporters contributes to pathogenesis of epilepsy. A numerous evident has shown glutamate uptake is a cellular process strictly dependent upon energy supply and a mitochondrial respiratory chain defect can induce a reduction of glutamate transport (see review (Danbolt, N. C. (2001) Prog Neurobiol 65, 1-105), MELAS (mitochondrial myopathy, encephalopathy, lactic acidosis and stroke-like episodes) is commonly associated with the A3243G mitochondrial DNA (mtDNA) mutation encoding the transfer RNA of leucine (UUR). DiFrancesco and his coworkers found a high relationship between A3243G mutation induced glutamate transport defect and mitochondrial ATP depletion in MELAS neurons (DiFrancesco, et al. (2008) Exp Neurol 212, 152-6), but the mechanism remains debate.

Na⁺, K⁺-ATPase plays a key role in the maintenance of the electrochemical gradient across the plasma membrane potentials and modulation of neurotransmitter release and uptake in the central nervous system (Stahl, & Harris (1986) Adv Neurol 44, 681-93). It has been demonstrated that inhibition of Na⁺, K⁺-ATPase activity increases Ca²⁺ entry into brain slices (Fujisawa, et al. (1965) Jpn J Pharmacol 15, 327-34) and glutamate release in the rat spinal cord (Li, S. & Stys, P. K. (2001) Neuroscience 107, 675-83), causes electrographically recorded seizures in mice (Jamme, et al. (1995) Neuroreport 7, 333-7). It has been found that a decreased Na⁺, K⁺-ATPase activity is in the post-mortem epileptic human brain (Grisar, T. (1984) Ann Neurol 16 Suppl, S128-34) and a mutation in the enzyme α-subunit gene associates with epilepsy in humans (Jurkat-Rott, et al. (2004) Neurology 62, 1857-61). Some results show there is a high correlation between the decreased of Na⁺, K+-ATPase activity and the duration of convulsive episodes (Souza, et al. (2009) Epilepsia 50, 811-23; Fighera, et al. (2006) Neurobiol Dis 22, 611-23). Recently, a research result implies that glutamate uptake in the brain is acutely regulated by the status of Na⁺, K⁺-ATPase and that glutamate transporter activity under direct control of Na⁺, K⁺-ATPase via the protein-protein interaction, in addition to the putative indirect reliance on Na³⁰, K⁺-ATPase through ion gradients. The study demonstrates that glutamate transporters and Na³⁰, K⁺-ATPase are part of the same macromolecular complexes and operate as a functional unit to regulate glutamatergic neurotransmission (Rose, et al. (2009) J Neurosci 29, 8143-55). Na⁺, K⁺-ATPase is present at high concentrations in brain, consuming 40-50% of the ATP generated in the organ (Erecinska & Silver (1994) Prog Neurobiol 43, 37-71) which activity is total depended on ATP supply. Although the exact mechanism of mitochondrial dysfunction inducing epilepsy is still unclear, our result proposes that a significant decreased Na⁺, K⁺-ATPase activity caused by mitochondrial dysfunction induced ATP depletion leading to glutamate transporters down-regulation may be one of the most important contributors to increase seizure susceptibility. Evidence also indicates that both of Na⁺, K⁺-ATPase and glutamate transporter are —SH contain proteins and sensitive to free radicals damage (Jamme, et al. (1995) Neuroreport 7, 333-7 ; Lees, G. J. (1993) Neuroscience 54, 287-322; Trotti, et al. (1998) Trends Pharmacol Sci 19, 328-34). Therefore, ROS may play a role in the reduction of the enzyme activity and transporters; although there is no significant increase ROS production was found in cytosol by our result in this study (Ting-Ting Wang group also didn't find any significant increased ROS production in cytosol).

The pathology damage of mitochondrial encephalopathy resultant by Sod2 mutant is same as that by mitochondrial DNA mutant, which indicates the pathogenesis of mitochondrial dysfunction may be common, regardless induced by primary mtDNA mutation or those factors may be secondary.

### MATERIALS AND METHODS

Animals Animal studies were carried out in accordance with the National Institute of Health Guide for the Care and Use of Laboratory Animals (NIH Publications No. 80-23). All procedures were approved by the Institute Animal Care and Use Committee (IACUC) of the University of Colorado Denver (UCD), which is fully accredited by the American Association for the Accreditation of Laboratory Animal Care. The mutant mice were monitored on a daily basis to get an accurate birth and death data. Pups were not culled or handled before P5 to avoid maternal rejection. Pups were genotyped at P5 by PCR as previously described (Li, et al. (1995) Nature Genet 11, 376-381).

Metalloporphyrin (AEOL11207) administration B6D2F1 Sod2−/− mice and their wild-type littermates (control mice) were treated with AEOL11207 (5 mg/kg) or vehicle by subcutaneous (s.c.) injection daily starting at 5 days of age until death or being sacrificed. AEOL11207 was dissolved in dimethyl sulfoxide (DMSO) and diluted with sterilized phosphate buffered saline (PBS) to achieve the desired final concentration (1% DMSO). The control animals were injected with sterilized PBS containing 1% DMSO. The animals were divided into four different groups: 1) control mice +vehicle; 2) B6D2F1 Sod2−/− mice+vehicle; 3) control mice +AEOL11207; 4) B6D2F1 Sod2−/− mice +AEOL11207. The treated and untreated mice were sacrificed at 15-16 days old for pathology and biochemistry assays or until death for survival and seize behavioral evaluation.

Behavioral seizure evaluation Positively confirmed Sod2^{-/-} mice aged P15-P24 that received either vehicle or AEOL 11207 treatment were video recorded (Q-See QD14B, Anaheim, Calif.) for a minimum of 8 hours a day in a custom designed observation cage. During the weaning period (P15-P18) mice were recorded in the presence of their mothers, and individually thereafter. Video was digitally recorded (Panasonic DMR-ES 15) and stored on DVD-R′s for observation and quantification of seizure number, duration, frequency, and severity by an observer blind to treatments. Seizure severity was scored according to the following scale: 1=immobilization and staring, 2=head-nodding, shaking, 3=forelimb tonic/clonic activity, 4=continuous forelimb tonic/clonic activity with falling, running or jumping. Due to the presence of abnormal gait and posturing in Sod2^{-/-} mice, only spontaneous motor seizures with scores>3 were included for comparison between groups. The scoring and analysis was conducted by an investigator blinded to genotype and treatment.

Determination of metalloporphyrin levels AEOL11207 was measured by HPLC-UV following previously described methods (Liang, et al. (2007) J Neurosci 27, 4326-33).

SOD2 activity assay SOD2 activity was measured in Sod2 mutant mice mitochondrial fractions from brain by the adrenochrome assay as described by Misra and Fridovich (1972) (Misra, & Fridovich, (1972) J Biol Chem 247, 3170-3175). The ability of SOD to inhibit the autoxidation of 0.3 mM epinephrine was measured in 50 mM sodium carbonate buffer, pH 10.2, 30° C. at 480 nm. Sodium cyanide (5 mM) was used to distinguish SOD2 activity.

Measurement of peroxynitrite-induced oxidation of dihydrorhodamine-123 inhibition by metalloporphyrins The peroxynitrite-dependent oxidation of dihydrorhodamine-123 to rhodamine-123 is measured based on previously described methods (Kooy et al. (1994) Free Radic Biol Med 16, 149-56; Szabo, et al. (1996) FEBS Lett 381, 82-6). Briefly, peroxynitrite (Canmy Inc) at 1 μM was added into 0.1 M phosphate buffered pH 7.4 containing 10 μM dihydrorhodamine 123 (Molecular Probes), in the absence or presence of metalloporphyrins (10 nM-100 μM). After 10 min incubation at room temperature, the fluorescence of rhodamine 123 was measured using a fluorimeter (Perkin-Elmer, Norwalk, Conn.) at an excitation wavelength of 500 nm, emission wavelength of 536 nm

Histochemical analyses The mice were sacrificed at 15-16 days old and brain paraffin sections (10 μm) were cut coronally and stained with Hematoxylin and Eosin (H&E) following the company protocol (Sigma, St. Louis Mo.). Fluoro-Jade B (Histo-Chem Inc., Jefferson, Ariz.) staining following previously described methods (Hopkins, et al. (2000) Brain Res 864, 69-80; Liang, et al. (2008) J Neurosci 28, 11550-6). Images were captured using a Nikon Optiphot-2 80i microscope equipped with epifluorescense optics (Nikon Inc., Melville, N.Y.). The Fluoro-Jade B positive signal of a given area was estimated with Image J (National Institutes of Health, Bethesda, Md.), an open source image manipulation tool, in three sections, 100 μm apart in the parietal cortex from both hemispheres of each animal. The average of the fluorescent relative density was expressed as percentage of the control.

Isolation mitochondrial fraction Mitochondria were isolated from the forebrain of mice according to the previously described methods (Liang & Patel, (2006) Free Radic Biol Med 40, 316-22). In brief, the forebrain was homogenized with a Dounce tissue grinder (Wheaton, Millville, N.J.) in mitochondrial isolation buffer (70 mM sucrose, 210 mM mannitol, 5 mM Tris HCl, 1 mM EDTA; pH 7.4). The suspensions centrifuged at 800 g 4° C. for 10 min. The supernatants were centrifuged at 13000 g 4° C. for 10 min., pellets washed with mitochondrial isolation buffer and centrifuged at 13000 g 4° C. for 10 min. to obtain crude mitochondrial fractions. The purity of mitochondrial fractions has been confined by immunoblot analysis of cytochrome c oxidase (COX) (EC1.9.3.1) subunit IV and lactate dehydrogenase (LDH) (EC 1.1.1.27) in mitochondrial and cytosolic fractions. There is no mitochondria contamination in cytosolic fraction and about 5-10% cytosol contamination in mitochondrial fraction (Liang & Patel, (2006) Free Radic Biol Med 40, 316-22).

Aconitase and fumarase activity assay Aconitase and fumarase activity are measured in mitochondrial fraction as previously described (Patel, et al. (1996) Neuron 16, 345-55).

Measurement of metabolomics by HPLC Ascorbate, cysteine, glutathione (GSH), glutathione disulfide (GSSG), methionine, tyrosine and 3-nitrotyrosine (3-NT) assay was performed with ESA (Chelmsford, Mass.) 5600 CoulArray HPLC equipped with eight electrochemical detector cells as previously described in the literature (Liang, et al. (2007) J Neurosci 27, 4326-33; Beal, et al. (1990) J Neurochem 55, 1327-39; Hensley, et al. (1998) J Neurosci 18, 8126-32). The potentials of the electrochemical detector are set at 0/150/300/450/570/690/780/850 mV. Analyte separation is conducted on a TOSOHAAS (Montgomeryville, Pa.) reverse-phase ODS 80-TM C-18 analytical column (4.6 mm×250 cm; 5 μm particle size). A two-component gradient elution system was used with component A of the mobile phase composed of 50 mM NaH₂PO₄pH 3.2, and component B composed of 50 mM NaH₂PO₄ and 40% methanol pH 3.2. The following gradient elution profile was used: 0-25 min, 100% A; 25-35 min, linear ramp to 50% B; 35-40 min, isocratic 50% B; 40-45 mM, linear ramp to 100% A; 45-50 min, isocratic 100% A at a flow rate of 0.6 ml/min. The samples prepared from the forebrain were sonicated in ice cold 0.1 M PCA and centrifuged at 16000 g 4° C. for 10 min. Aliquots (50 μl) of the supernatant was injected to HPLC. The level of 3-NT was expressed as a ratio of 3-NT to tyrosine.

Measurement of Reduced CoA (CoASH) and its GSH disulfide (CoASSG) CoASH and CoASSG were measured by HPLC equipped with UV detection as previously described (Liang & Patel, (2006) Free Radic Biol Med 40, 316-22).

Immunoblot analysis of glutamate transporter Glutamate transporter immunoblot analysis was followed the protocol described before (Liang & Patel, (2004) Free Radic Biol Med 36, 542-54). The primary antibody was used with rabbit against GLT-1 (1:5000: Abcom Inc. Cambridge, Mass.). The bands were scanned on a Storm Optical Scanner (Molecular Dynamics Inc. Sunnyvale, Calif.) and quantative analysis of each band was performed by ImageQuant software (Amersham Biosciences, Buckinghamshire, England). The ratios of GLT-1 to β-actin were calculated from each mouse, the mean ratios in Sod2+/+ group were designated as 100%.

Measurement of AMP, ADP and ATP by HPLC The forebrains were dissected out, quickly frozen with liquid nitrogen, weighed and sonicated in 10% w/v (e.g. 20 mg/200 μl) 0.42 M perchloric acid. (The homogenates can be store at −80 ° C.). The homogenates were centrifuge at 13000 g 4° C. for 15 min. 100 μl supernatant was removed to a new tube and neutralized with 10 μl 4 N KOH. The neutralized supernatant was mixed well and left on −20° C. for at least 10 min to ensure removal of perchlorate (as KClO₄). After centrifugation at 8500g 4 ° C. for 10 min, mix same volume (100 μl) supernatants and same volume (100 μl) 50 mM KH₂PO₄, an aliquot of 50 μl of the mixture was injected into the HPLC system. The levels of AMP, ADP and ATP are quantified by HPLC-UV set at 258 nm following previous described (Sellevold, et al. (1986) J Mol Cell Cardiol 18, 517-27; Botker, et al. (1994) J Mol Cell Cardiol 26, 41-8). Analyte are separated by 5 μM, 4.6×250 cm C-18 reversed-phase column. Mobile phase is composed of 50 mM KH₂PO₄, 10% methanol, 3 mM tetrabutyl ammonium sulphate (TBAS), pH 6.0 and flow rate set at 0.8 ml/min.

Na³⁰, K⁺-ATPase activity assay The ATPase activities in brain homogenates were determined by measuring the amount of inorganic phosphate released from the substrate ATP according to a previously described colorimetric method (Lanzetta, et al. (1979) Anal Biochem 100, 95-7; Chen, et al. (2007) Basic Clin Pharmacol Toxicol 101, 108-16). The brain tissues were completely sonicated and centrifuged at 13000 g at 4° C. for 10 min ˜25 μg protein of the supernatant was incubated at 37±0.5° C. for 15 min in 300 μl of NaCl 100 mM, KCl 20 mM, MgCl₂ 5 mM, Tris-HCl 30 mM, ethyleneglycol bis (amino-ethylether) tetraacetate (EGTA), 0.5 mM, glucose 20 mM at pH 7.4 and ATP 5 mM. Reactions were terminated by the addition of 150 μl of a solution containing ammonium molybdate (1.05%) in 0.5 N HCl. The optical density at 340 nm was determined by a plate reader. The absorbance values obtained were converted to activity values by linear regression using a standard curve for sodium monobasic phosphate included in the assay at various concentrations. Inorganic phosphate released (in nmol) was taken to represent the concentration of inorganic phosphate released by the enzymatic hydrolysis of ATP. Na⁺, K⁺-ATPase activity was determined by subtracting 1 mM ouabain insensitive Mg²⁺-ATPase activity from total Na³⁰, K⁺-ATPase activities.

Statistical analyses Survival analysis was performed using the Kaplan-Meier method. For all biochemical analyses, two-way ANOVA was used. P values less than 0.05 were considered significant.

What is claimed is:

1. A method of treating a mitochondrial disorder comprising administering to a subject in need thereof a therapeutically effective amount of a metalloporphyrin compound.

2. The method of claim 1, wherein the subject is a child.

3. The method of claim 1, wherein the mitochondrial disorder is epilepsy.

4. The method of claim 3, wherein the subject has temporal lobe epilepsy or other acquired epilepsies comprising acute or chronic epilepsies arising from pathological insult.

5. The method of claim 4, wherein the acute or chronic epilepsies comprise acute or chronic epilepsies arising from hypoxia, trauma, viral infections, fever, alcohol withdrawal or aging which increase oxidative stress and mitochondrial disorder.

6. The method of claim 3, wherein said method reduces the frequency or severity of epileptic seizures of said subject.

7. The method of claim 1, wherein the mitochondrial disorder is an acute or chronic neurological disorder.

8. The method of claim 1, wherein the subject has an inherited mitochondrial disease or inherited epilepsies.

9. The method of claim 2, wherein the subject has pediatric epilepsies, encephalopathies or pediatric movement disorders.

10. The method of claim 9, wherein the pediatric movement disorders are derived from fever, trauma, metabolic deficiencies, genetic abnormalities, chromosomal abnormalities, hypoxic/ischemic episodes or combination thereof.

11. The method of claim 1, wherein the metalloporphyrin compound has the formula: 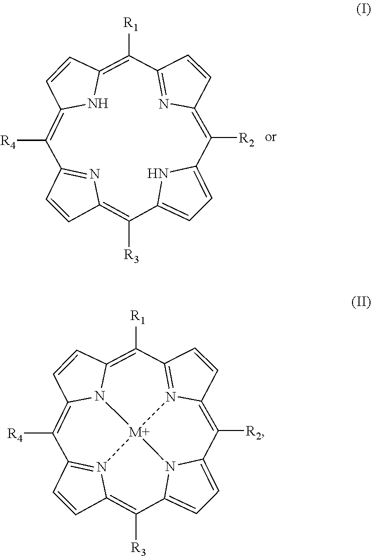
wherein R₁, R₂, R₃, and R₄ are each independently —CF₃, —CO₂R₈, —COR_{8′}, 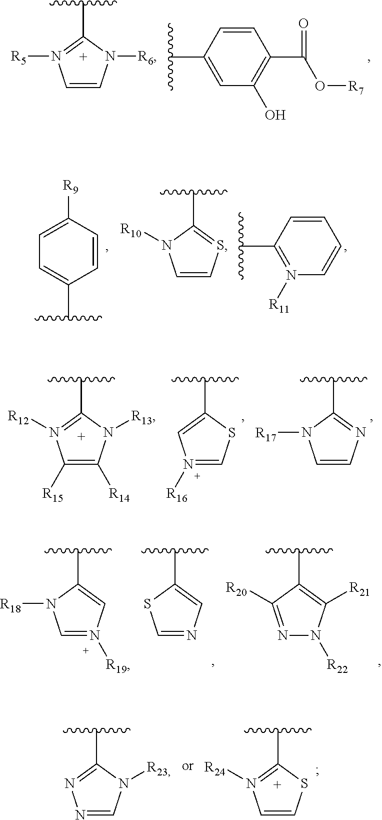
R₅, R₆, R₇, R₈, R_{8′}, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄ are each independently hydrogen, halogen, —CN, —CF₃, —OH, —NH₂, —COOH, —COOR₂₅, —CH₂COOR₂₅, —CH₂COOH, an unsubstituted or substituted alkyl, unsubstituted or substituted heteroalkyl, unsubstituted or substituted cycloalkyl, unsubstituted or substituted heterocycloalkyl, unsubstituted or substituted aryl, or an unsubstituted or substituted heteroaryl; R₂₅ is an unsubstituted alkyl; and M is a metal.

12. The method of claim 11, wherein R₂₅ is C₁-₁₀ alkyl.

13. The method of claim 12, wherein R₂₅ is —CH₃ or a C₁₋₅ alkyl.

14. The method of claim 11, wherein the metal is manganese, iron, cobalt, copper, nickel, or zinc.

15. The method of claim 11, wherein R₁, R₂, R₃, and R₄ are 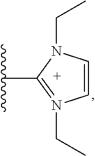
and the metal is manganese.

16. The method of claim 11, wherein R₁ and R₃ are —CO₂—CH₃, R₂ and R₄ are —CF₃, and the metal is manganese.

17. The method of claim 11, wherein R₁ and R₃ are 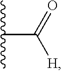
R₂ and R₄ are 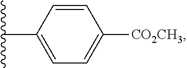
and the metal is manganese.
